(19) United States
(12) Patent Application Publication
(10) Pub. No.: US 2024/0185990 A1
(43) Pub. Date: Jun. 6, 2024

(54) System and Method for Processing Medical Image Data

(71) Applicant: Blackford Analysis Ltd., Edinburgh (GB)

(72) Inventor: Keith HOUSTON, Birmingham (GB)

(21) Appl. No.: 18/075,820

(22) Filed: Dec. 6, 2022

(51) Int. Cl. G16H 30/40; G16H 15/00; G16H 30/20

(52) U.S. Cl. CPC G16H 30/40; G16H 15/00; G16H 30/20

(57) ABSTRACT

A system, method and computer program product for processing medical data from a clinical software application. Medical image data is processed by a clinical software application and the processed data is then mapped to a proposed report tree. A plurality of tagged nodes are generated in the proposed report tree by tagging data nodes within the processed medical data with a corresponding semantic tag. The proposed report tree is displayed through a viewer application and user input approving or rejecting the proposed report tree is received through the viewer application. A final report tree is generated based on the received user input and stored in an electronic data management system.

### FIELD

The present disclosure relates generally to processing medical image data, and in particular to generating reports of medical image data.

### INTRODUCTION

The following is not an admission that anything discussed below is part of the prior art or part of the common general knowledge of a person skilled in the art.

Electronic medical data is generated about patients during various interactions with a medical organization. Medical data is often collected when a patient is imaged using an image acquisition device. Medical image data can be used for various clinical purposes, such as assessing, diagnosing and monitoring the progression of a disease or injury.

It is often desirable to perform image processing and/or automated analysis of medical image data before presenting the medical images to clinicians. The image processing may be intended to support a clinician's review of the scan by, for example, quantifying clinically relevant features in the images, or adding new medical images that are derived in some way from the original medical image or images and may provide clinically useful data.

Various different types of clinical software applications can be used to generate processed medical image data. These applications generate processed medical data of various different types and using different formats or arrangements for the processed data. Accurately and efficiently interpreting the processed medical data can provide valuable insights for clinicians in assessing a patient.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In accordance with this disclosure, systems, methods and computer program products are provided that can facilitate the visualization and review of processed medical data from clinical software applications. In particular, processed medical data from a plurality of different clinical software applications can be mapped to a consistent report structure. The processed medical data can then be displayed to a user through a viewer application as a proposed report defined in accordance with the report structure. The user can then provide simple inputs approving or rejecting portions of the report in order to define a final report. The final report can then be stored for subsequent review by a clinician, e.g. in a PACS, RIS or other data management or reporting system. This can simplify and streamline a user's review of processed medical data from different clinical software applications by ensuring a consistent representation of the processed medical data regardless of the clinical software applications used to generate the data.

In an aspect of this disclosure, there is provided a method for processing medical data received from a clinical software application, the method comprising: receiving, at a processor, a set of processed medical data generated by the clinical software application, wherein the set of processed medical data is generated by the clinical software application based on at least one medical image generated by an enterprise imaging device and associated image data corresponding to the at least one medical image, wherein the at least one medical image is associated with a medical image study; mapping, by the processor, the set of processed medical data to at least one proposed report tree, wherein each proposed report tree is defined with a hierarchical tree structure that includes a plurality of data nodes arranged into one or more sub-trees, wherein each data node is associated with a corresponding subset of the processed medical data; for each proposed report tree, generating a plurality of tagged nodes by tagging, by the processor, at least some of the data nodes within that proposed report tree with a corresponding semantic tag, wherein each semantic tag specifies a semantic meaning of a corresponding subset of the processed medical data associated with the corresponding tagged node; displaying, by the processor, a given proposed report tree from the at least one proposed report tree through a viewer application, wherein the given proposed report tree is displayed with at least some of the tagged nodes visible within the viewer application; receiving, by the processor, user input through the viewer application in response to the proposed report tree being displayed through the viewer application, wherein the user input includes an approval or rejection of at least a portion of the hierarchical tree structure; generating, by the processor, a final report tree based on the received user input; and storing, by the processor, the processed medical data contained within the final report tree in an electronic data management system.

The final report tree can omit any portion of the hierarchical tree structure that was rejected by the user input whereby the processed medical data stored in the electronic data management system omits the processed medical data that was rejected by the user input.

The user input can define the approval or rejection of each data node within the hierarchical tree structure.

The user input can include a report tree finalization input, and each data node within the hierarchical tree structure can be accepted except for those data nodes that are explicitly rejected by a user.

The given proposed report tree can include at least one dependent node nested within a corresponding parent node within the hierarchical tree structure. When the given proposed report tree is displayed in the viewer application the nested dependent node can be initially hidden.

The given proposed report tree can be displayed within the viewer application with a user-selectable input usable to render the nested dependent node visible.

The given proposed report tree can be displayed within the viewer application with at least one user input prompt, where each user input prompt corresponds to a particular data node, and each user input prompt can enable a user to provide a corresponding input through the viewer application accepting or rejecting the definition of the corresponding particular data node.

A given user input prompt can enable the user to accept or reject the definition of the corresponding particular data node and any portion of a sub-tree that is dependent on that corresponding particular data node.

The set of processed medical data generated by the clinical software application can include at least one DICOM Structured Report object, and each DICOM Structured Report object can be mapped to a different corresponding proposed report tree.

The set of processed medical data generated by the clinical software application can include a DICOM Structured Report object, and the plurality of data nodes can be identified from a defined report format associated with the DICOM Structured Report object.

The method can include storing the processed medical data contained within the final report tree in the electronic data management system by: generating a final DICOM Structured Report object using the final report tree; and storing the final DICOM Structured Report object in the electronic data management system.

The set of processed medical data corresponding to the given proposed report tree can include one or more unstructured DICOM objects. The one or more unstructured DICOM objects can be mapped to data nodes within the proposed report tree using a hierarchical structure that includes a plurality of node levels, the plurality of node levels including a study node level, a series node level, and an instance node level. Each data node can be tagged based on the node level that data node is mapped to.

The set of processed medical data can include one or more unstructured DICOM objects and each unstructured DICOM object can be mapped to a corresponding data node within the proposed report tree based on the content of the processed medical data associated with that unstructured DICOM object.

The method can include mapping the set of processed medical data to the at least one proposed report tree by mapping the set of processed medical data to the hierarchical tree structure using an application independent mapping.

The method can include mapping the set of processed medical data to the at least one proposed report tree by: determining an application specific mapping corresponding to the clinical software application; and mapping the set of processed medical data to the hierarchical tree structure according to the application specific mapping.

The application specific mapping can be determined by: identifying the clinical software application that generated the set of processed medical data; and selecting the application specific mapping from amongst a plurality of potential application specific mappings, where the application specific mapping is selected as the potential application specific mapping associated with the identified clinical software application.

The method can include modifying the application specific mapping based on the received user input.

In accordance with an aspect of this disclosure, there is provided a system for processing medical data received from a clinical software application, the system comprising: a network device; and one or more processors in communication with the network device, the one or more processors configured to: receive, using the network device, a set of processed medical data generated by the clinical software application, wherein the set of processed medical data is generated by the clinical software application based on at least one medical image generated by an enterprise imaging device and associated image data corresponding to the at least one medical image, wherein the at least one medical image is associated with a medical image study; map the set of processed medical data to at least one proposed report tree, wherein each proposed report tree is defined with a hierarchical tree structure that includes a plurality of data nodes arranged into one or more sub-trees, wherein each data node is associated with a corresponding subset of the processed medical data; for each proposed report tree, generate a plurality of tagged nodes by tagging at least some of the data nodes within that proposed report tree with a corresponding semantic tag, wherein each semantic tag specifies a semantic meaning of a corresponding subset of the processed medical data associated with the corresponding tagged node; display a given proposed report tree from the at least one proposed report tree through a viewer application, wherein the given proposed report tree is displayed with at least a given proposed report tree from the at least one proposed report tree through the viewer application, wherein the given proposed report tree is displayed with at least some of the tagged nodes visible within the viewer application; receive user input through the viewer application in response to the proposed report tree being displayed through the viewer application, wherein the user input includes an approval or rejection of at least a portion of the hierarchical tree structure; generate a final report tree based on the received user input; and store, using the network device, the processed medical data contained within the final report tree in the electronic data management system.

The one or more processors can generate the final report tree to omit any portion of the hierarchical tree structure that was rejected by the user input such that the processed medical data stored in the electronic data management system omits the processed medical data that was rejected by the user input.

The user input can define the approval or rejection of each data node within the hierarchical tree structure.

The user input can include a report tree finalization input, and each data node within the hierarchical tree structure can be accepted except for those data nodes that are explicitly rejected by a user.

The given proposed report tree can include at least one dependent node nested within a corresponding parent node within the hierarchical tree structure. The one or more processors can be configured to display the given proposed report tree in the viewer application with the nested dependent node initially hidden.

The one or more processors can be configured to display the given proposed report tree through the viewer application with a user-selectable input usable to render the nested dependent node visible.

The one or more processors can be configured to display the given proposed report tree through the viewer application with at least one user input prompt, where each user input prompt corresponds to a particular data node, and each user input prompt enables a user to provide a corresponding input through the viewer application accepting or rejecting the definition of the corresponding particular data node.

A given user input prompt can enable the user to accept or reject the definition of the corresponding particular data node and any portion of a sub-tree that is dependent on that corresponding particular data node.

The set of processed medical data generated by the clinical software application can include at least one DICOM Structured Report object, and one or more processors can be configured to map each DICOM Structured Report object to a different corresponding proposed report tree.

The set of processed medical data generated by the clinical software application can include a DICOM Structured Report object, and the one or more processors can be configured to identify the plurality of data nodes from a defined report format associated with the DICOM Structured Report object.

The one or more processors can be configured to store the processed medical data contained within the final report tree in the electronic data management system by: generating a final DICOM Structured Report object using the final report tree; and storing the final DICOM Structured Report object in the electronic data management system.

The set of processed medical data corresponding to the given proposed report tree can include one or more unstructured DICOM objects. The one or more processors can be configured to store the processed medical data contained within the final report tree in the electronic data management system by storing the unstructured DICOM objects corresponding to the final report tree in the electronic data management system.

The set of processed medical data can include one or more unstructured DICOM objects. The one or more processors can be configured to: map the one or more unstructured DICOM objects to data nodes within the proposed report tree using a hierarchical structure that includes a plurality of node levels, the plurality of node levels including a study node level, a series node level, and an instance node level; and tag each data node based on the node level that data node is mapped to.

The set of processed medical data can include one or more unstructured DICOM objects. The one or more processors can be configured to map each unstructured DICOM object to a corresponding data node within the proposed report tree based on the content of the processed medical data associated with that unstructured DICOM object.

The one or more processors can be configured to map the set of processed medical data to the at least one proposed report tree by: mapping the set of processed medical data to the hierarchical tree structure using an application independent mapping.

The one or more processors can be configured to map the set of processed medical data to the at least on proposed report tree by: determining an application specific mapping corresponding to the clinical software application; and mapping the set of processed medical data to the hierarchical tree structure according to the application specific mapping.

The one or more processors can be configured to determine the application specific mapping by: identifying the clinical software application that generated the set of processed medical data; and selecting the application specific mapping from amongst a plurality of potential application specific mappings, wherein the application specific mapping is selected as the potential application specific mapping associated with the identified clinical software application.

The one or more processors can be configured to modify the application specific mapping based on the received user input.

In accordance with an aspect of this disclosure, there is provided a non-transitory computer-readable medium with instructions stored thereon for processing medical data received from a clinical software application, that when executed by a processor, performs the method as described herein.

It will be appreciated by a person skilled in the art that a system, device, method or computer program product disclosed herein may embody any one or more of the features contained herein and that the features may be used in any particular combination or sub-combination. Other features and advantages of the present application will become apparent from the following detailed description taken together with the accompanying drawings. It should be understood, however, that the detailed description and the specific examples are given by way of illustration only, since various changes and modifications within the scope of the application will become apparent to those skilled in the art from this detailed description.

### BRIEF DESCRIPTION OF THE DRAWINGS

For a better understanding of the various examples described herein, and to show more clearly how these various examples may be carried into effect, reference will be made, by way of example, to the accompanying drawings which show at least one example, and which are now described. The drawings are not intended to limit the scope of the teachings described herein.

FIG. 1 is a block diagram of an example medical data processing system.

FIG. 2 is a block diagram of an example data processing device that may be used in the medical data processing system of FIG. 1.

FIG. 3 is a flowchart illustrating an example method for processing medical image data.

FIG. 4 is a flowchart illustrating an example method for generating a finalized report.

FIG. 5 is a block diagram showing an example report tree structure.

FIG. 6 is an example of processed medical data output by a clinical application.

FIG. 7 is a visual representation of an example mapping of the processed medical data shown in FIG. 6 to a report tree structure.

FIG. 8 is another example of processed medical data output by a clinical application.

FIG. 9 is a visual representation of an example mapping of the processed medical data shown in FIG. 8 to a report tree structure.

FIG. 10A is a screenshot of an example viewer application showing a proposed report tree with example nodes in a collapsed state.

FIG. 10B is a screenshot of the example viewer application showing the proposed report tree of FIG. 10A with the example nodes in an expanded state.

FIG. 10C is a screenshot of the example viewer application showing a different medical image included in the proposed report tree of FIG. 10A.

FIG. 10D is a screenshot of the example viewer application showing another medical image included in the proposed report tree of FIG. 10A.

FIG. 10E is a screenshot of the example viewer application showing a node acceptance screen for the proposed report tree of FIG. 10A.

FIG. 11 is a screenshot of the example viewer application showing an example of another proposed report tree.

FIG. 12 is a visual representation of the example mapping shown in FIG. 7 illustrating an example of accepted and rejected data nodes.

FIG. 13 is a visual representation of the example mapping shown in FIG. 9 illustrating an example of accepted and rejected data nodes.

### DETAILED DESCRIPTION

Various apparatuses or methods will be described below to provide an example of the claimed subject matter. No example described below limits any claimed subject matter and any claimed subject matter may cover methods or apparatuses that differ from those described below. The claimed subject matter is not limited to apparatuses or methods having all of the features of any one apparatus or methods described below or to features common to multiple or all of the apparatuses or methods described below. It is possible that an apparatus or methods described below is not an example that is recited in any claimed subject matter. Any subject matter disclosed in an apparatus or methods described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It should also be noted that the terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal or a mechanical element depending on the particular context. Furthermore, the term “communicative coupling” indicates that an element or device can electrically, optically, or wirelessly send data to another element or device as well as receive data from another element or device.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g. 112a, or 1121). Multiple elements herein may be identified by part numbers that share a base number in common and that differ by their suffixes (e.g. 1121, 1122, and 1123). All elements with a common base number may be referred to collectively or generically using the base number without a suffix (e.g. 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g. a keyboard, mouse, a touchscreen, and the like), and at least one output device (e.g. a display screen, a printer, a wireless radio, and the like) depending on the nature of the device. For example and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “DICOM” refers to the Digital Imaging and Communications in Medicine (DICOM) standard for the communication and management of medical imaging information and related data as published by the National Electrical Manufacturers Association (NEMA).

As described herein, “HL7” refers to the Health Level 7 (HL7) standard as published by Health Level Seven International.

As used herein, the term “patient” generally refers to any human or animal or other subject that may undergo an imaging exam to acquire medical image data from that human or animal.

As described herein, “medical images”, “image data”, or “images” refers to image data collected by image acquisition devices. The images are visual representations of an area of body anatomy that may be used for various purposes such as clinical analysis, diagnosis and/or medical interventions, commonly referred to as radiology. These images can include images of removed organs and tissues and/or images of an area of anatomy captured in-situ. Medical imaging can also establish a database of normal anatomy and physiology to make it possible to identify abnormalities.

Imaging exams often taken place when a patient interacts with a medical organization. Medical imaging data is generated when the patient is imaged by an image acquisition device during the imaging exam. This medical imaging data generally includes image data (also referred to as image pixel data or pixel data) and associated image acquisition metadata.

Medical imaging data can be generated using various different types of image acquisition devices. Each image acquisition device is associated with a specific imaging modality. A “modality” refers to the categorical type of image acquisition generated by different image acquisition devices (e.g. CT, MR, x-ray, ultrasound or PET; as in, “those images have modality ‘CT’”). The different modalities or types of image acquisition devices refer to the technology used to create the image. Examples of imaging modalities include various imaging technologies including X-ray Plain Film (PF), digital X-rays, Cardiology imaging devices, Computed Tomography (CT) images, ultrasound images, nuclear medicine imaging including Positron-Emission Tomography (PET), Veterinary imaging devices, Magnetic Resonance Imaging (MRI) images, mammographic images, or any other standardized images used in a medical organization. The medical images may be collected using analog means such as film and then subsequently digitized, or may be constructed from data originally collected using digital sensor means such as a Charge Coupled Device (CCD) and processed into image data. The image data may be provided in JPEG, JFIF, JPEG2000, Exif, GIF, BMP, PNG, PPM, PGM, PBM, PNM, WebP, HDR, HEIF, or any other known format. The image data may be provided in an uncompressed image format, in a lossless compressed format, or in a lossy compressed format.

The medical imaging data from an individual imaging exam is referred to as a study or imaging study. Characteristics of an imaging study such as format, size and number of images generated by an individual imaging exam (also referred to as a scan or a scanning episode) can vary based on numerous factors such as the imaging modality type, the clinical purpose for the imaging exam (e.g. the diagnostic or clinical need for the imaging test), the part of the body (the area of anatomy) being scanned etc.

An individual imaging exam can capture a single image, a series of images of a single area of anatomy, multiple series of images covering a single area of anatomy, multiple series of images covering multiple different areas of anatomy, a video of an area of anatomy etc. An individual imaging exam may capture multiple series of images in various circumstances, such as when the image acquisition device is operable in multiple modes of operation (e.g. an MR scanner) or to acquire images before and after administration of a contrast agent for example. In some cases, an imaging exam may acquire medical imaging data in the form of video imaging data (e.g. a video of a beating heart). In many cases, video imaging data can be treated as a series of images for the purposes of data processing and/or analysis.

Medical imaging data can assist a clinician in evaluating and monitoring a patient, e.g. to diagnose, assess and monitor the state or progression of a condition such as a disease or injury. It is often desirable to perform additional processing and/or analysis on medical imaging data before the medical imaging data is presented to a clinician to support the clinician's review of the scan. Such processing and/or analysis can be performed by a clinical software application and can include, for example, quantifying clinically relevant features in the images, or adding new, clinically useful, image data derived in some way from the originals.

As described herein, clinical software applications (also referred to as clinical applications) are software applications that generate additional information about a medical study or studies through the analysis of image data (including pixel data) and/or image metadata of one or more images of the study or studies. Such analysis might range from simple to highly complex calculations including statistical inferences and machine learning. The clinical software application analysis may add clinically relevant information and/or probabilistic information such as confidence levels, probabilities, and/or certainties. Alternatively or in addition, the clinical software application may generate new medical image data based on transformations, enhancements, or other operations on the original image data.

The resulting data generated by a clinical software application (also referred to as processed medical data) can take various forms, such as one or more new images or image regions, modified images or image regions, and/or additional metadata derived from the original images. Examples of image processing and analysis that may be performed by a clinical software application include image compression, data encryption, information enhancement, image registration, anatomy recognition, contrast detection, image quality, quantification of brain abnormalities and changes from MR images in order to track disease evolution, liver iron concentration analysis from MR images of the liver, and the removal of personally identifying information among many others.

Clinical software applications can process medical data in an automated manner using various techniques including machine learning and artificial intelligence (AI) techniques. AI-based clinical application can enhance analysis of image data and provide advanced insights such as screening for cancer, aiding in the diagnosis of neurological diseases and identifying fractures and other musculoskeletal injuries.

The processed medical data output from a clinical software applications is often reviewed to ensure that clinically relevant information is presented. For example, the processed medical data may initially be reviewed by a technician. The technician can manually correct errors or make changes to the processed medical data before it is presented to the clinician.

However, reviewing processed medical data from different clinical software applications may not be straightforward. For instance, the results from an AI-based clinical application are often complex and difficult to interpret. The format of the results can also vary depending on the clinical software application being used. As a result, a user needs to know how to interpret different data formats to properly review and interpret the application results for different clinical software applications. This can involve significant and ongoing training for users and can cause the review process to be inefficient and unreliable.

The results from clinical software applications are often presented to a user in a dense, text-based format that makes review challenging and unintuitive. The text-based format is often non-interactive and does not allow a user to easily make changes or correct errors. The structure of the data presented is also often inconsistent between different clinical software applications. Manually reviewing complex reports to correct errors or make changes can waste time and introduce the possibility of human error, particularly when dealing with a variety of report formats.

In medical data processing systems, the image data and image metadata associated with an imaging study is typically stored in a DICOM format. The DICOM image metadata typically includes information about how the medical image data was acquired, such as scanner data, anatomy data (e.g. the body regions imaged), image spatial data (e.g. spatial information about pixels and slice spacings), study identifier data, image identifier data, acquisition date, acquisition time etc.

Clinical software applications also typically use the DICOM format when outputting processed medical data. These clinical software applications can generate reports that include the processed medical data in a manner consistent with the DICOM format. Reports are often encoded using either a DICOM Structured Report object or an unstructured DICOM object.

Structured Report objects are typically formatted in an organized, tree-like structure. Nodes within these Structured Report objects may further contain cross-linked information. Unstructured objects simply contain the imaging data and quantitative results generated by a clinical software application. The existing formats of these reports often require technicians to spend unnecessary time reviewing and updating the reports. While the defined DICOM formats provide some level of standardization in data output, the formats allow for variations in how data is presented between different clinical software applications. As a result, a technician will still need familiarity with a variety of data formats in order to effectively review processed medical data from multiple clinical software applications.

The present disclosure provides systems, methods and computer program products that enable processed medical data (including structured report objects and unstructured report objects) to be received, automatically organized, reviewed, and stored in a consistent and efficient manner. The present disclosure enables the visualization and review of processed medical data through a consistent and simplified interface to allow proposed reports to be reviewed, accepted and/or rejected.

Image data from an imaging modality can be sent to a clinical software application for processing. The processed medical data generated by the clinical software application can be sent to a report tree generation engine. The report tree generation engine can map the processed medical data to a proposed report tree. Tagged nodes can be generated for the data nodes in the proposed report tree. The proposed report tree can be displayed through a viewer application in a consistent format regardless of the initial form of the processed medical data. A user can provide input approving or rejecting portions of the proposed report tree through the viewer application. A final report tree can be generated based on the user input and then stored in an electronic data management system such as a PACS, RIS or data reporting system.

The processed medical data can be mapped to a proposed report tree using a hierarchical tree structure. The hierarchical tree structure can be a universal hierarchical tree structure that is applied to processed medical data from a plurality of different clinical software applications. Mapping the processed medical data to a universal hierarchical tree structure can standardize the results from different clinical software applications and make the process of viewing and editing reports more consistent and efficient.

An example hierarchical tree structure can define a hierarchy of node levels that includes a report node (which can be the highest level parent node), one or more group nodes, one or more finding nodes, and one or more property nodes (which can be the lowest level child nodes). Each report node can contain one or more group nodes; each group node can contain one or more finding nodes; and each finding node can contain one or more property nodes. Each data node can be associated with a corresponding subset of the processed medical data.

Optionally, the processed medical data can be mapped to a proposed report tree using an application independent mapping. That is, the processed medical data coming from multiple clinical software applications can be mapped using the same mapping process.

Alternatively, the processed medical data can be mapped to a proposed report tree using an application specific mapping. In this configuration, the platform can determine an application specific mapping corresponding to the clinical software application and map the processed medical data according to the application specific mapping. The application specific mapping can be determined by identifying the clinical software application that generated the set of processed medical data and selecting the application specific mapping associated with the identified clinical software application. This configuration allows the platform to tailor mapping processes for each clinical software application, refining the mapping accuracy and accounting for idiosyncrasies in how certain applications represent processed medical data.

The platform can also update the application specific mappings on an ongoing basis in response to user interactions with proposed report trees. For example, machine learning models can be used to update and modify the application specific mappings of the proposed report trees. Supervised learning models can be trained on the accept and reject behavior data over time to learn patterns in the users' accept and reject inputs. These patterns can inform improved application specific mappings that generate more accurate results. Unsupervised learning models can also be used to predict the best mappings of the proposed report trees.

The data nodes of the proposed report tree can be tagged with semantic tags that specify a semantic meaning of the subset of the processed medical data associated with the corresponding tagged node. The semantic tags can guide the viewer experience by defining where and how the tagged nodes are displayed in a viewer application.

The proposed report tree can be displayed through a viewer application with the tagged nodes visible. A user can view the proposed report tree through a viewer application shown on a physical display. Various information related to the proposed report tree can be displayed in the viewer application. For example, the viewer application can display the image data related to the proposed report tree, other processed medical data related to the proposed report tree, group and finding icons for easier navigation, toggling functionalities for the reports and findings and editing options.

While the user is viewing the proposed report tree in the viewer application, they can provide input in response to the proposed report tree. The user input can include an approval or rejection of at least a portion of the hierarchical tree structure, comments on the proposed report tree, and/or various other edits to the proposed report tree. The viewer application can include input buttons associated with each data node allowing a user to easily accept or reject the data associated with each node.

Once a user has finished reviewing the processed medical data, the user can proceed to confirm the accepted and rejected inputs. The viewer application can include a report finalization input to allow the user to initiate a report tree confirmation. Once the user initiates the report tree confirmation, the viewer application can display a confirmation prompt that provides a summary of all the accepted and rejected data nodes. By proceeding through the confirmation prompt, the user can save a final report tree which can be later reviewed by a clinician as part of evaluating a patient's interaction with the medical organization.

The final report tree can be generated based on the received user input. The final report tree can omit portions of the hierarchical tree structure that were rejected by the clinician and retain portions or the hierarchical tree structure that were accepted by the clinician. The final report tree and the processed medical data contained within the final report tree can be stored in a medical data storage system such as a PACS, RIS, or other data storage and/or reporting system.

Depending on the nature of the electronic data management system, the final report tree may be defined or stored in different formats. For example, the final report tree can be stored in a PACS in a DICOM format. Alternatively or in addition, the final report tree can be converted into one or more report messages defined in a format compatible with the electronic data management system. For example, the report messages can be formatted according to the HL7 v2.x standard or converted into FHIR objects. The report messages can also be defined to call proprietary APIs exposed by the electronic data management system (e.g. a reporting system or RIS).

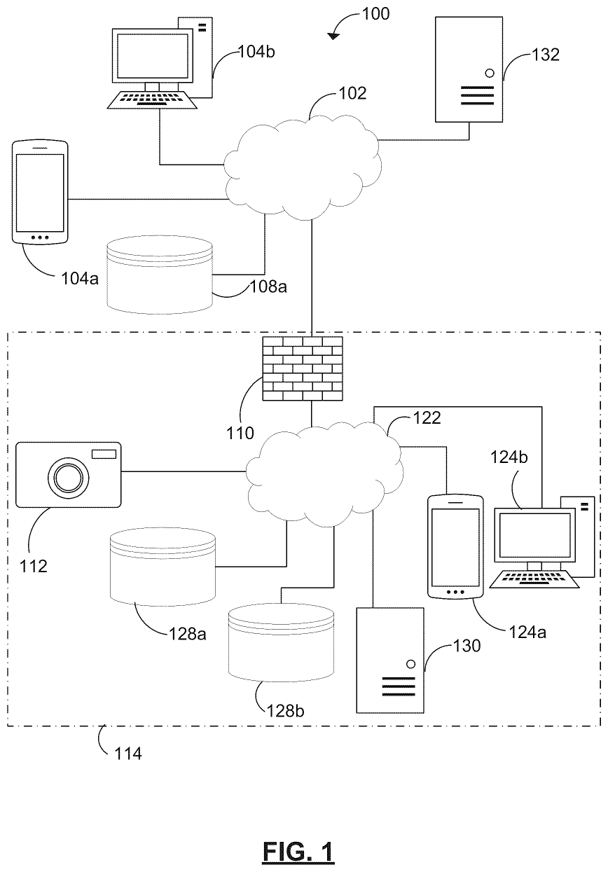

Reference is first made to FIG. 1, showing an example medical data processing system 100. The example system 100 includes a plurality of user devices 104 such as mobile device 104a and computer device 104b, a network 102, a server 132, and an imaging device in the form of an enterprise image management device 108a.

As illustrated, system 100 includes a medical organization 114. Medical organization 114 has an associated medical organization network 122 that is connected to network 102 by a firewall 110. A plurality of medical organization user devices 124 (e.g. mobile device 124a and computer device 124b) may operate within the medical organization network 122. The medical organization user devices 124 and the plurality of user devices 104 can generally be provided using various types of computing devices, as discussed below. The medical organization network 122 can also include a server 130 (e.g. a processing server), imaging devices in the form of one or more enterprise image management devices 128a and 128b and one or more image acquisition devices 112. The server 132 and enterprise image management device 108a may be referred to, respectively, as a remote server 132 and a remote enterprise image management device 108a in the sense that they are external to the medical organization network 122.

Alternatively, the processing server 130 may be external to the medical organization 114 and the enterprise image management devices 128 may forward image data and associated metadata to the external processing server via network 122, firewall 110, and network 102.

User devices 104 and medical organization user devices 124 may be used by end users to access an application (not shown) running on remote server 132 over network 102 and network 122. For example, the application may be a web application, or a client/server application. The user devices 104 and medical user devices 124 may be desktop computers, mobile devices, or laptop computers. The user devices 104 and medical user devices 124 may display the web application, and may allow a user to review medical data, including medical images and image metadata, from medical organization 114. A user of user devices 104 and medical organization devices 124 may be a technician or clinician user at medical organization 114 who may review the medical data, including processed medical data from the clinical software applications. A user may be a radiologist whose role is to review (or read) medical images, or a referring clinician (for example, the non-radiologist clinician who referred the patient for a scan) who may receive a report from the radiologist.

The users at user devices 104 and medical organization devices 124 may include an administrator user who may administer the configuration of clinical software applications or mapping rules for medical organization 114.

Enterprise image management devices may include both remote enterprise image management device 108a, first enterprise image management device 128a inside the medical organization 114, and second enterprise image management device 128b inside the medical organization 114. While a single remote enterprise image management device 108a is shown, it is understood that there may be a plurality of remote enterprise image management devices. Similarly, while only two enterprise image management device 128a and 128b are shown, it is understood that there may be more enterprise image management devices.

The enterprise image management devices may be a Picture Archiving and Communication System (PACS) server, a Modality Worklist (MWL), a medical image data archive or another enterprise image management device. For example, an enterprise image management device may be an IntelePACS® from Intelerad®, an IntelliSpace® PACS from Philips®, an enterprise image management device such as the Enterprise Imaging Solution® suite from Change Healthcare®, a Medicor® MiPACS® Modality Worklist or an IBM iConnect Enterprise Archive.

Remote enterprise image management device 108a may be located remotely from the medical organization 114. There may be one or more remote enterprise image management devices 108a. For example, a remote PACS may be at an affiliated medical organization to the medical organization 114, for example, a satellite clinic. A remote enterprise image management device 108a may provide economical storage and convenient access to medical images and image metadata from multiple image acquisition devices external to medical organization 114. A PACS may support live Query/Retrieve, archive Query/Retrieve, be configured to auto-forward, or a combination of these roles.

Remote enterprise image management device 108a may be a Modality Worklist (MWL), where the MWL makes patient demographic information from a Radiology Information System (RIS) available at an image acquisition device, and providing, amongst other things, a worklist of patients who will attend the image acquisition device for imaging in the near future. The MWL may further provide in-progress studies and completed studies.

Remote enterprise image management device 108a may be a remote image acquisition device configured using auto-forward to the medical organization 114.

The remote enterprise image management device 108a may store image metadata in a DICOM format, an HL7 format, an XML-based format, or any other format for exchanging image data and associated metadata.

Remote server 132 may be a commercial off-the-shelf server. Alternatively, the remote server 132 may be a server running on Amazon® Web Services (AWS®) or another similar hosting service. The remote server 132 may be a physical server, or may be a virtual server running on a shared host. The remote server 132 may have an application server, a web server, a database server, or a combination thereof. The application server may be one such as Apache Tomcat, etc. as is known. The web server may be a web server for static web assets, such as Apache® HTTP Server, etc. as is known. The database server may store user information including structured data sets, electronic form mappings, and other electronic form information. The database server may be a Structured Query Language (SQL) such as PostgreSQL® or MySQL® or a not only SQL (NoSQL) database such as MongoDB®.

The remote server 132 is in communication with processing server 130, and may provide data analysis for the processing server, or data aggregation in conjunction with processing server 130. The remote server 132 may provide a web-based interface for browsing analysis of the medical data and the clinical software applications running at processing server 130.

Alternatively, the remote server 132 may be provided inside the medical organization 114 and connected to the processing server 130 via network 122.

Alternatively, there may be more than one server providing the functionality of remote server 132, including one inside the medical organization 114 in communication with processing server 130 via network 122, and a second remote server accessible to the medical organization 114 via network 122, firewall 110, and network 102.

Network 102 may be a communication network such as the Internet, a Wide-Area Network (WAN), a Local-Area Network (LAN), or another type of network. Network 102 may include a point-to-point connection, or another communications connection between two nodes.

Network 122 may be a communication network such as an enterprise intranet, a Wide-Area Network (WAN), a Local-Area Network (LAN), or another type of network. Network 122 may include a point-to-point connection, or another communications connection between two nodes. The network 122 may exist at a single geographical location, or may span multiple geographical locations. Network 122 is separated from network 102 by firewall 110, which may provide for network address translation for the medical organization 114, virtual private network access for the remote user devices 104, access control lists for network traffic sent between network 122 and network 102 (and vice-versa).

Medical organization 114 may include one or more related medical organizations that share medical image data and associated metadata over one or more networks. The one or more related medical organizations may include one or more image acquisition devices, one or more geographical locations, a plurality of clinician users, a plurality of administrative users, and a plurality of patients attending the one or more image acquisition devices for medical imaging services.

The medical organization 114 has a firewall 110, one or more image acquisition devices 112, a first enterprise image management device 128a, a second enterprise image management device 128b, a processing server 130, one or more medical user devices 124 such as mobile device 124a and computer device 124b, and medical network 122.

Firewall 110 may be a commercial off-the-shelf network firewall that performs network translation and routing between network 102 and network 122. For example, firewall 110 may be a Cisco® firewall, a Sonicwall® firewall, or another firewall as is known.

The one or more image acquisition devices 112 may include a variety of different imaging modalities that generate medical images such as X-ray Plain Film (PF) devices, digital X-ray devices, Computed Tomography (CT) devices, ultrasound devices, nuclear medicine imaging devices including Positron-Emission Tomography (PET) devices, Magnetic Resonance Imaging (MRI) devices, mammographic devices, or any other imaging modality used in a medical organization. The one or more image acquisition devices 112 may include mobile imaging devices such as mobile CT scanners. The medical images generated by the one or more imaging devices may be collected using analog means such as film and then subsequently scanned or may initially be collected using digital sensor means such as a Charge Coupled Device (CCD). The one or more image acquisition devices 112 may operate to produce studies of patients of the medical organization 114. The one or more image acquisition devices 112 may collect various metadata at the time it captures images of the patient. The metadata collected by the one or more image acquisition devices 112 may be in DICOM format, HL7 format, or other formats for image data and associated metadata formats as are known. The one or more image acquisition devices 112 may include, for example, a General Electric® (GER) Revolution Apex® CT image acquisition device, a Siemens® Magnetom Vida® MR image acquisition device, and a Canon® UltiMax® x-ray image acquisition device.

The first enterprise image management device 128a and the second enterprise image management device 128b may be PACS. Alternatively, either the first or the second enterprise image management devices 128 may be a Modality Worklist at one of the one or more image acquisition devices 112. The enterprise image management device 128 may store medical image data collected at the one or more image acquisition devices 112, and image metadata corresponding to the medical image data.

The image data generated by the one or more image acquisition devices 112 and stored in the first enterprise image management device 128a and second enterprise image management device 128b may be provided in JPEG, lossless JPEG, Run-Length Encoding (RLE), JFIF, JPEG2000, Exif, GIF, BMP, PNG, PPM, PGM, PBM, PNM, WebP, HDR, HEIF, or any other known image format.

Processing server 130 may be a commercial off-the-shelf server system. The processing server 130 may be configured to execute a software platform that manages the processing of medical images, executes one or more clinical software applications to process the medical images, and further generates reports by mapping the processed medical data to a defined report tree structure. The processing server 130 may be a physical server, or a virtual server running on a shared host. The processing server 130 can be a single server, or multiple different individual servers configured to work cooperatively to provide the platform application, the one or more clinical software applications, the report generation engine and a viewer application. The processing server 130 is connected via network 122 to the enterprise image management devices 128, the one or more image acquisition devices 112, and medical user devices 124. The processing server 130 is further in network communication with the remote enterprise image management device 108a and remote server 132 via network 122, firewall 110, and network 102.

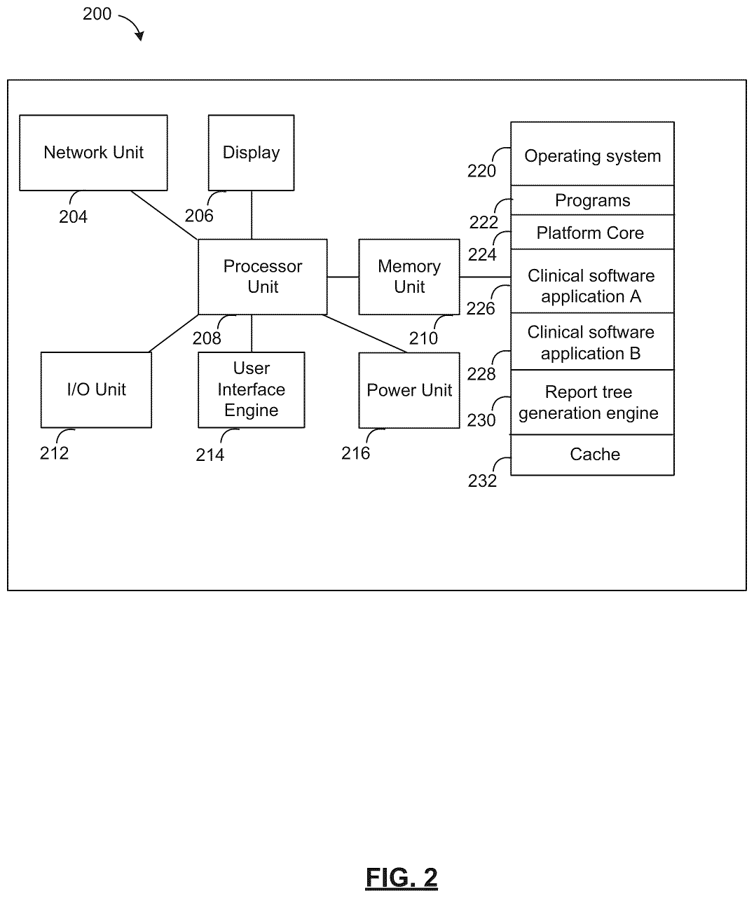

User devices, such as the processing server 130, can be implemented using a combination of hardware and software components as described further in FIG. 2.

Reference is next made to FIG. 2, showing an example block diagram of a medical data processing device 200. Medical data processing system 200 is an example medical data processing system that may be used in a medical data processing system such as processing server 130 in system 100. The medical data processing system 200 is connected to a network through network unit 204.

Medical data processing system 200 can include a network unit 204, an output device such as a display or speakers 206, an input unit such as mouse or keyboard, a processor 208 (also referred to as a processing unit), a memory unit 210, and a power unit 216.

The network unit 204 may be a standard network adapter such as an Ethernet or 802.11x adapter. The memory unit 210 can include both volatile memory and non-volatile storage memory. The power unit 216 can provide power to the processing server 130. Medical data processing system 200 can include an I/O unit 212 that provides access to server devices including disks and peripherals. The I/O hardware can provide local storage access to the programs running on processing unit 208.

The processor unit 208 may include a standard or special-purpose processor. Alternatively, or in addition, a plurality of processors can be used by the processor unit 208 and may function in parallel. Alternatively, or in addition, a plurality of processors can be used by the processor unit 208 including a Central Processing Unit (CPU) and a Graphics Processing Unit (GPU). There may be a plurality of CPUs and a plurality of GPUs.

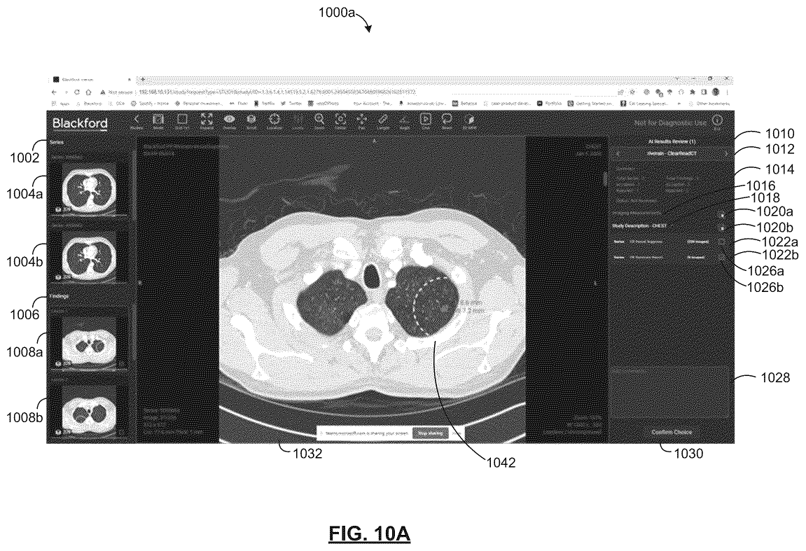

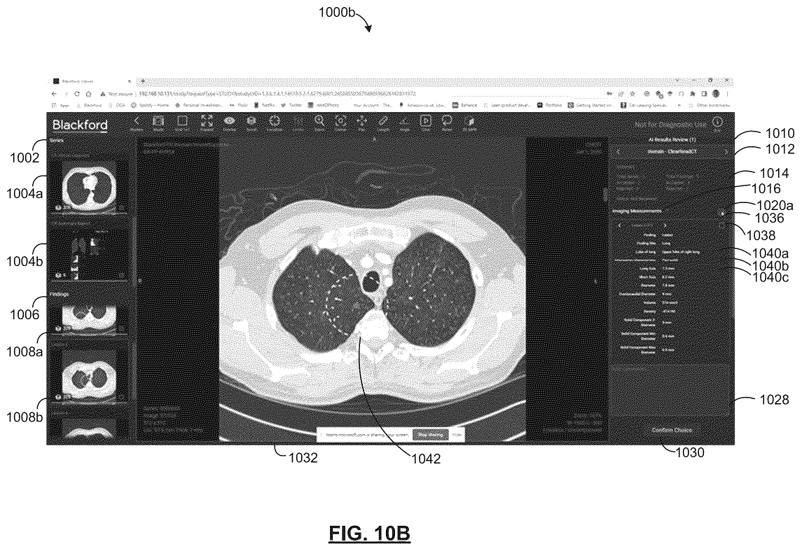

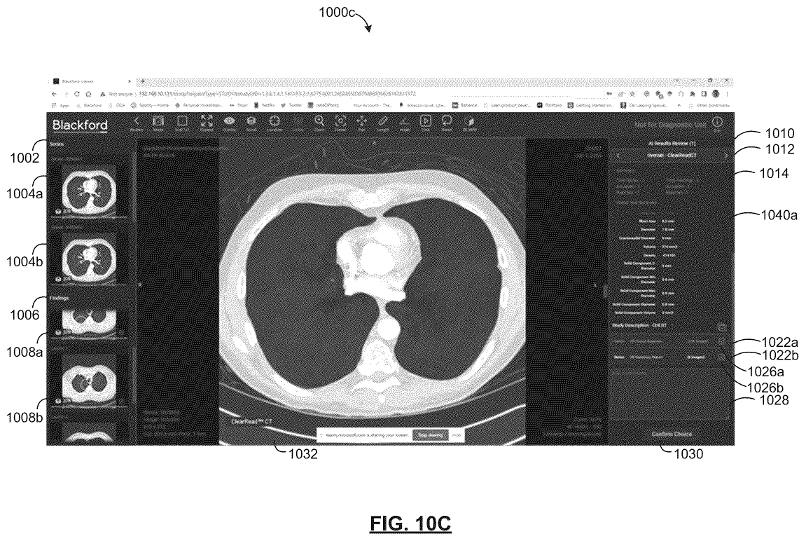

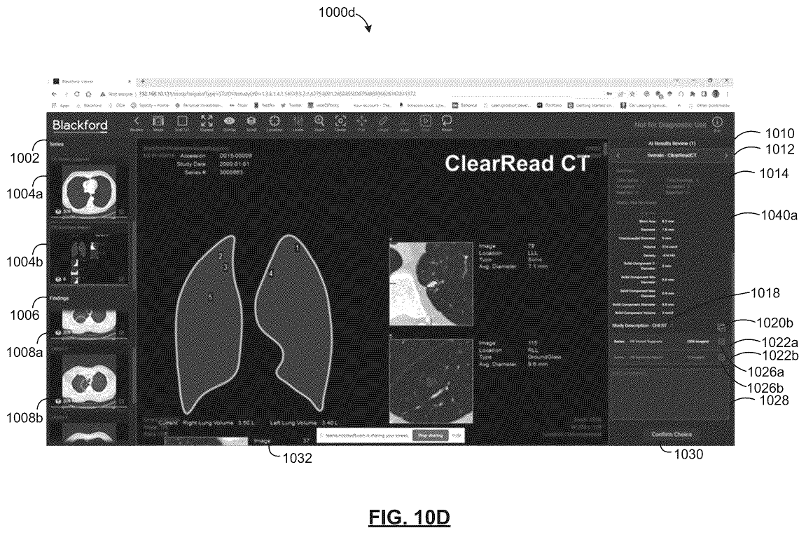

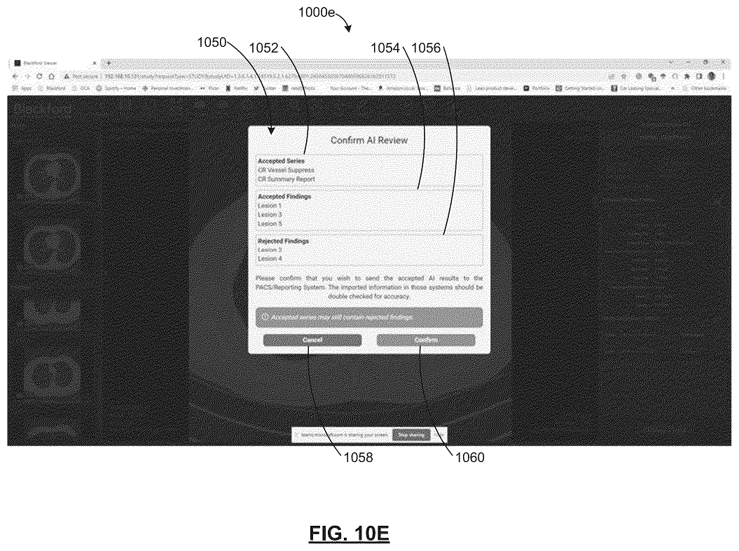

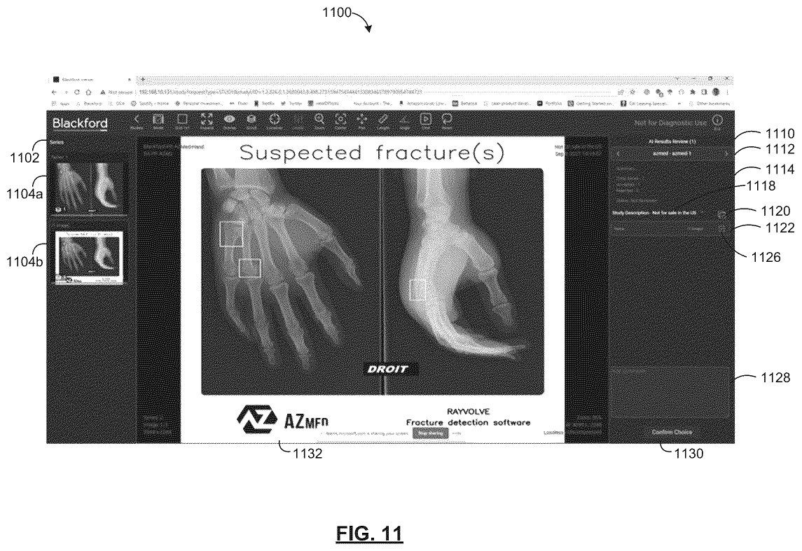

The processor unit 208 can also execute a user interface engine 214 that is used to generate various GUIs (see e.g. FIGS. 10-11). The GUIs can be presented to users of the user devices 104 and 124 to allow the users to review data such as medical images, proposed report trees, input clinical requests and/or report approvals/modifications, and interact with data generated by the image processing system. The information submitted using these interfaces may be processed by a report tree generation engine 230. The user interface engine 214 may be an Application Programming Interface (API) or a Web-based application that is accessible via the network unit or a graphical user interface application accessed via remote desktop or a similar type of user interface engine.

The memory unit 210 can store instructions executable by the processor to implement an operating system and one or more applications. The memory unit 210 can store instructions for an operating system 220, various programs 222, a platform core 224, one or more clinical software applications 226 and 228, a report tree generation engine 230, and a cache 232.

The operating system 220 may be a Microsoft Windows Server operating system, or a Linux-based operating system, or another operating system.

The programs 222 comprise program code that, when executed, configures the processor unit to operate in a particular manner to implement various functions and tools for the processing server.

The platform core 224 provides functionality for managing the medical data processing system 200. This may include providing an API available via the network unit for administration of the processing server, including configuration of the clinical software applications and structure report generation engine. Alternatively, a user may access a web interface of the platform core 224 via the user interface engine 214 and/or the network unit 204 to administer the medical data processing system 200.

The platform core 224 may be deployed to a physical server or a virtual server on the same network as its host medical organization. For example, the platform core 224 may be deployed as a Windows® Service. The platform core 224 may include a settings editor that allows an administrator to configure the platform core, or other components.

The memory unit 210 can also store data usable by the applications and engines operating on the device. For example, the memory unit can store one or more databases and/or caches 232 that can be used for long-term (or at least non-volatile) storage of data such as study data, image data, series data, metadata, patient data, order data, report data etc. The databases can also store reference data accessible by components of the imaging processing system such as the report tree generation engine 230. For example, the reference data may include a configuration file, a configuration model, one or more report trees etc.

The network unit 204 connects the processing server to the medical organization networks using a variety of protocols and modes of operation. The network interface 204 may receive medical image data and associated image metadata using a push mode of operation, where image devices send data to the processing server. Alternatively or in addition, the medical image processing system 200 may actively fetch medical image data and associated metadata from imaging devices and/or storage components. The network unit 204 may send a polling request to one or more imaging devices or image storage components using a “pull” mode of operation of the medical image data and associated metadata. The network unit 204 may receive medical images and image metadata using both a pull and a push mode of operation from the image devices.

The data cache 232 is a software component that can be used to assemble received image data. The data cache 232 may cache both medical images and image metadata.

For example, data cache 232 may store image data and/or associated metadata following a first query sent by the platform 200. When a second query is transmitted by a component of the platform 200, the data cache 232 can provide a locally cached copy of the image data and/or associated metadata to the requesting component. In this manner, the data cache 232 may provide image data and associated metadata to the components of the platform 200 without the need for a query request to be sent using network unit 204.

The cache 232 can include a storage portion in memory where medical images, image metadata, study data, study metadata, series data, series metadata, processed medical data etc. may be stored as the processing server is generating a report tree. Study data, study metadata, series data, series metadata, image data, and image metadata may be received by the processing server 200 at the network unit 204 and then stored in the cache 232.

The cache 232 may hold image data and associated metadata until all relevant data is available for processing or analysis by an application 226 or 228 or by report generation engine 230. Alternatively or in addition, the data cache 232 may cache all received data for a specified caching period. As a result, any study, series or image data need only be fetched once over the network within the specified caching period. This may allow the cached data to be used by several different clinical software applications without requiring separate data transmissions over the network.

Optionally, data cache 232 may include two or more distinct caches. For example, cache 232 may include a first cache for image data and a second cache for report tree data. This may simplify data management, as the image data may tend to be large in size while the associated report tree data may be smaller in size. Accordingly, different data management protocols may be applied to the data stored in the image cache as opposed to the data stored in the report tree cache.

The data cache 232 may be implemented using an off-the-shelf caching program such as memcached, redis, or another caching application as is known. The data cache 232 may also provide for read through and lazy loading functionality such that data is only loaded into the data cache 232 as needed, e.g. upon request of other components of the processing system 232.

The one or more clinical software applications 226 and 228 provide data processing capabilities. Each of the clinical software applications 226 and 228 is a software application that accepts study data, performs some processing on the data and generates processed medical data (e.g. a processed study) that is sent to the report tree generation engine 230. While only two clinical software applications are shown in FIG. 2, it is understood that there may be any number of clinical software applications, and potentially many more clinical software applications.

Clinical software applications 226 and 228 can automatically process medical data to create processed data including medical images and metadata. Examples of clinical software applications 226 and 228 include an image registration application, a registration application, an anatomy recognition application, a contrast detection application, an image quality application, a Personal Health Information (PHI) Removal application, a quantification application (e.g. of brain abnormalities and/or changes between studies and/or an estimate of liver-iron-concentration from a study of the liver) and so forth.

The clinical software applications 226 and 228 may run in a process at processing server 200. Alternatively, the clinical software applications 226 and 228 may run in a virtual machine (e.g. using VMware ESX) or a container (e.g. using Docker) at processing server 200. Alternatively, the clinical software applications 226 and 228 may be located separately from processing server 200 in network communication with processing server 200, and in such a case the study data may be sent to the clinical software application using network unit 204, and the processed study may be received back from the clinical software application using network unit 204. Alternatively, a combination of clinical software application hosting may be used, for example a first clinical software application in one or more clinical software applications may run in a process at a processing server 200, a second clinical software application in the one or more clinical software applications may be located separately from the processing server 200, and a third clinical software application in the one or more clinical software applications may be located in a virtual machine or container at processing server 200.

Alternatively, the clinical software applications 226 and 228 may be software modules provided via a remote cloud-hosted service which are accessed by using a remote API.

The report tree generation engine 230 can receive processed medical data from the clinical software applications 226 and 228. For example, the report tree generation engine 230 can receive a processed study (i.e. the processed medical data) formatted as a DICOM Structured Report object or a DICOM unstructured object from the clinical software applications. A DICOM unstructured object may also be referred to as a DICOM instance and may include images. In some cases, the report tree generation engine 230 may receive the processed medical data after it is stored in the cache 232.

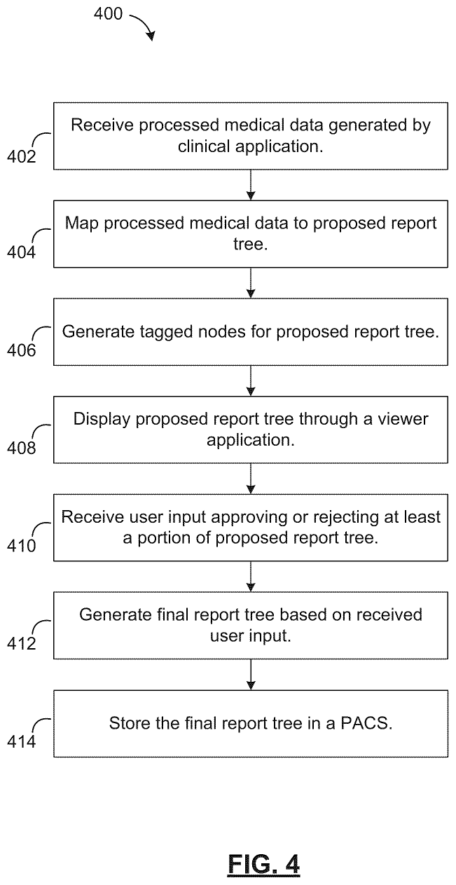

The report tree generation engine 230 can generate a proposed report tree from the processed medical data (i.e. results from the clinical software application(s)). The report tree generation engine 230 can process the processed medical data from each clinical software application independently. That is, for each clinical application, the report tree generation engine 230 can transform the result data from that clinical software application into a proposed report tree. An example process of generating a report tree that can be implemented by the report tree generation engine 230 is shown in FIG. 4 and described in further detail below.

In general, the report tree generation engine 230 can generate a proposed report tree based on the processed medical data received from a clinical software application. The proposed report tree can be defined according to a hierarchical tree structure. For example, the hierarchical tree structure can be defined with node levels including a study level, a series level, and an instance level. The report tree generation engine 230 can allocate the data nodes in received processed medical data to the various levels of the hierarchical tree structure in order to define a proposed report tree.

The report tree generation engine 230 can generate a proposed report tree for unstructured processed medical data by sorting all unstructured objects into nodes of the hierarchical tree structure. Similarly, the report tree generation engine 230 can generate a proposed report tree for structured processed medical data (e.g. a DICOM Structured Report object) by mapping the data nodes from that object to the hierarchical structure.

The report tree generation engine 230 can also tag the nodes within a proposed report tree with semantic tags. Each node can be tagged with a corresponding semantic tag. Each semantic tag can specify a semantic meaning of the processed medical data associated with the corresponding tagged node.

The tagged proposed report tree can be displayed to a user on display 206. User interface engine 214 can generate a visualization of the tagged proposed report tree that can be shown through a viewer application on display 206. A user can then provide input through the viewer application in response to the displayed proposed report tree.

The user interface engine 214 can define various user input prompts (e.g. selectable buttons) displayable within the viewer application. A user can interact with the input prompts to provide feedback on the proposed report tree. The user inputs can include inputs accepting or rejecting various portions of the proposed report tree including the entire proposed report tree, individual report tree nodes and/or subtrees of the proposed report tree. When the user provides inputs confirming a finalized report tree, the report tree generation engine 230 can generate a final report tree.

The final report tree can omit all rejected nodes from the proposed report tree. The final report tree can then be stored in an electronic data management system such as a PACS, RIS, or other data storage and/or reporting system. The report tree generation engine 230 can send the final report tree to the data management system for storage through network unit 204. The final report tree can be subsequently retrieved and reviewed by the same or a different user, such as a clinician for example.

The report tree generation engine 230 can define the final report tree using various data formats, such as DICOM for example. In some cases, the final report tree can be stored directly in the data management system in the original report format. For example, the final report tree can be stored in a PACS in a DICOM format.

Alternatively or in addition, the final report tree can be converted into an alternative format prior to being transmitted to, or stored by, an electronic data management system. For example, the final report tree can be converted into one or more report messages defined in a format compatible with the electronic data management system. For example, the report message(s) can be formatted according to the HL7 v2.x standard or converted into FHIR objects. Alternatively or in addition, the report message(s) can be defined to call proprietary APIs exposed by the electronic data management system (e.g. a reporting system or RIS).

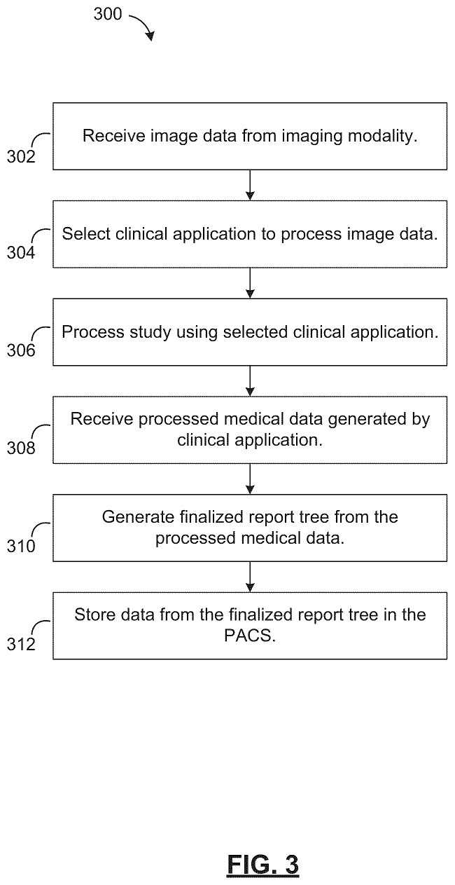

Referring now to FIG. 3, shown therein is an example method 300 of processing medical images. Method 300 is an example of a method for processing a plurality of medical images that can generate a finalized report tree from the processed medical data received from clinical applications. Method 300 may be implemented using various image processing systems, such as the example processing system 200 described herein above.

At 302, image data can be received from the imaging modality. The imaging data may be received from an enterprise image management device. Alternatively, the imaging data may be received directly from an image acquisition device. The image data can include associated metadata and one or more series.

The image study metadata can include a plurality of metadata elements. The image study metadata can provide information relating to various aspects of the image data. The image study metadata elements can include a study instance identifier (or UID), a study date, a study time, a referring physician's name, a study identifier, a unique patient identifier (e.g. an accession number), a study description, a referenced study sequence, a referenced SOP class identifier (or UID), and a reference SOP instance identifier (or UID). Other image study metadata may include an admitting diagnosis description, a patient age, and a patient weight.

The image study can be sent to the processing device by various image devices, such as a PACS server or MWL server. Receipt of the study can be triggered in various ways, e.g. in response to collection of the study at an image acquisition device, or in response to a user input. For example, a user may manually initiate a transmission of the study data to the processing system. Alternatively or in addition, study data may be transmitted to the processing system automatically upon collection by the image acquisition device (or receipt by the enterprise image management device). The study can be pushed (or auto-forwarded) to the processing system from a PACS server or a MWL server, or alternatively from an image acquisition device.

Alternatively or in addition, the processing system may initiate retrieval of the study through a pull mode of operation from the image device. The processing system can send a polling request to one or more imaging devices (e.g. a PACS server and/or an MWL server). The corresponding imaging device can response to the polling request by transmitting the current study data to the processing system.

At 304, one or more clinical applications can be selected to process image data. The one or more clinical application can be selected based on an automatic selection rule or on a manual selection input. An automatic selection rule may be based on the nature of the image data. For example, the image data received from a particular imaging modality may be sent to the same clinical application each time. Alternatively or in addition, the user may specify the clinical application for processing the image data through a user input.

Examples of processes for routing medical data to one or more clinical applications are described in further detail in PCT Patent Application No. PCT/EP2020/083558 entitled “SYSTEMS AND METHODS FOR PROCESSING MEDICAL IMAGES USING RELEVANCY RULES” and U.S. patent application Ser. No. 17/750,138 entitled “SYSTEMS AND METHODS FOR ROUTING MEDICAL IMAGES USING ORDER DATA”, the entirety of each of which is incorporated herein by reference.

At 306, the image study can be processed using the selected clinical application identified at 304. The processing system 200 can process the image study using the selected clinical software application to generate a processed study. The processed study can include one or more of processed patient metadata, processed imaging data and processed order data (i.e. study metadata, study image data, or study order data that was generated or modified as a result of the processing performed by the selected clinical software application).

The selected clinical application can automatically process the image study (i.e. without intervention by a clinician, technician or other user). That is, steps 302, 304 and 306 may be performed without requiring manual intervention by a user.

At 308, the processed medical data generated by the clinical application can be received. The processed study can be received in a DICOM format. The processed study can be defined as a DICOM Structured Report object or unstructured object.

At 310, a finalized report tree can be generated from the processed medical data. An example process 400 for generating a finalized report tree is described in further detail herein below with reference to FIG. 4.

In general, the finalized report tree can be generated by mapping the processed medical data (from 308) to a proposed report tree. The proposed report tree can be defined according to a hierarchical tree structure in which data notes are allocated to specified node levels within the tree structure. Each of the data nodes can be tagged with a corresponding semantic tag. The proposed report tree can then be displayed to a user through a viewer application. The user can interact with the viewer application to approve or reject at least a portion of the proposed report tree. The finalized report tree can be generated in response to the user inputs received through the viewer application.

At 312, the data from the finalized report tree can be stored in a data management system such as a PACS, RIS, and/or other data management and/or reporting system. The finalized report tree can be stored in the data management system with the originating image study for future retrieval, viewing and editing.

The final report tree can be transmitted and/or stored using various data formats. In some cases, the final report tree can be stored directly in the data management system in the original report format. For example, the final report tree can be stored in a PACS in a DICOM format.

Alternatively or in addition, the final report tree can be converted into an alternative format prior to being transmitted to, or stored by, an electronic data management system. For example, the final report tree can be converted into one or more report messages defined in a format compatible with the electronic data management system. For example, the report message(s) can be formatted according to the HL7 v2.x standard or converted into FHIR objects. Alternatively or in addition, the report message(s) can be defined to call proprietary APIs exposed by the electronic data management system (e.g. a reporting system or RIS).

Referring now to FIG. 4, shown therein is an example method 400 of generating a report tree. Method 400 is an example of a method for processing medical data generated by a clinical application in which the processed medical data can be organized into a consistent tree structure. Method 400 may be implemented using various image processing systems, such as the example processing systems 100 and 250 described herein above.

At 402, processed medical data generated by the clinical application can be received by the processing unit. The processed medical data can be generated by the clinical software application based on at least one medical image generated by an enterprise imaging device and associated image data corresponding to the at least one medical image. The medical image can be associated with a medical image study.

The clinical application can produce processed medical data, i.e. new information in addition to the original image data. The result data can take various forms, such as one or more new images or image regions, modified images or image regions, and/or additional metadata derived from the original images. Examples of image processing and analysis that may be performed by a clinical software application include image compression, data encryption, information enhancement, image registration, anatomy recognition, contrast detection, image quality, quantification of brain abnormalities and changes from MR images in order to track disease evolution, liver iron concentration analysis from MR images of the liver, and the removal of personally identifying information among many others. For instance, a clinical application may output coordinate data related to a mammography image to indicate areas with increased likelihood of breast cancer.

The processed medical data can be output by the clinical software application in different formats and structures. For example, the processed medical data can be defined as a DICOM Structured Report object or an unstructured DICOM object.

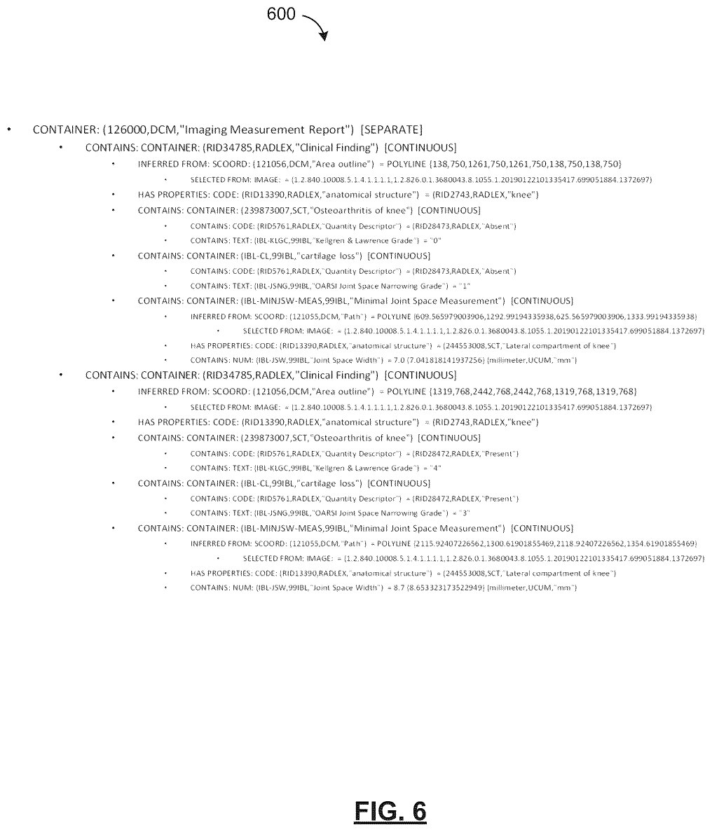

An example of a DICOM Structured Report object is shown in FIG. 6. The DICOM Structured Report object 600 shown in FIG. 6 is an example of processed medical data that may be generated by a clinical software application.

As shown in FIG. 6, the DICOM Structured Report object 600 can include a plurality of nested containers. Each container can include a subset of the processed medical data.

The entire report can be contained within a top-level container referred to as a report container (e.g. an Imaging Measurement Report). All of the processed medical data of the Structured Report object 600 can be contained within the top-level container and/or a container nested therein.

Each report can also include one or more clinical findings. Accordingly, one or more finding containers can be nested within the report container. A clinical finding can be a clinical investigation result item and may include normal/abnormal observations, judgements, or assessments of patients. Examples of clinical findings include: cyanosis, increased blood pressure, pulmonary nodules, pericardial effusion, extrahepatic biliary dilation, normal breast tissue, lymphadenopathy, bowel dilation, osseous lesions, etc.

In some cases, a report can include a plurality of findings and associated finding containers. Each finding container can be nested below the top-level report container. For example, the Structured Report object 600 includes two finding containers.

Each clinical finding can include one or more properties. Accordingly, one or more property containers can be nested within each finding container. The properties can provide detailed information about the associated clinical finding (i.e. the clinical finding that property is nested within). For example, properties can explain the data from which the finding was inferred, the anatomical structure of interest, grading scores, measurement data or any other property of a clinical findings.

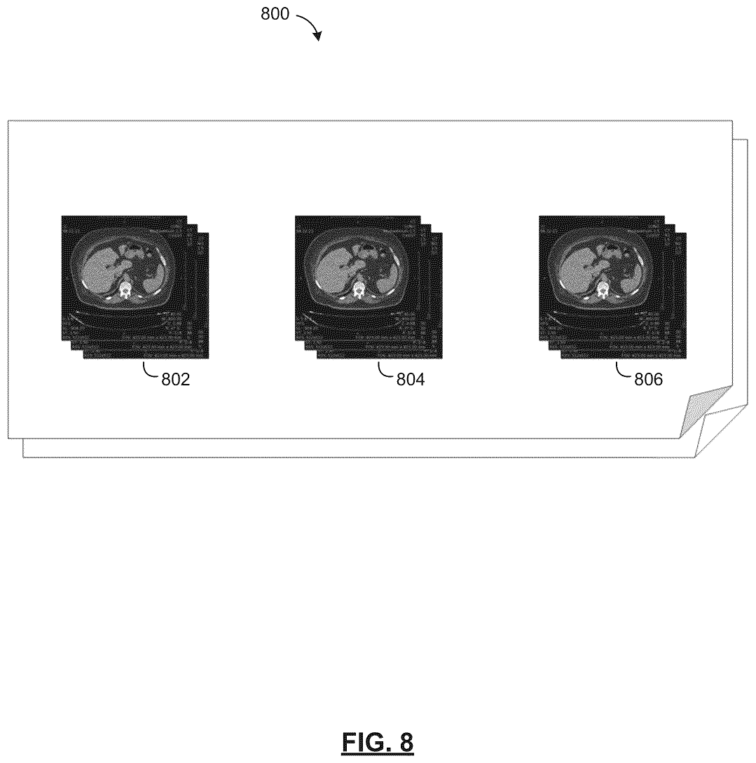

In processing medical data, clinical software applications may also generate non-Structured objects as shown in FIG. 8. The unstructured processed medical data may not correspond to, or be arranged into, a report by the clinical software application. As shown in the example of FIG. 8, the unstructured processed medical data can include a plurality of image instances 802, 804 and 806. The example unstructured data includes an image study containing several image series of spinal cord cross sections captured through a CT scan. As compared with the structured object 600, it can be seen that the processed medical data in unstructured data 800 has not been organized into any defined structure.

At 404, the processed medical data can be mapped to a proposed report tree. The proposed report tree can be defined according to a specified hierarchical tree structure. The hierarchical tree structure can define a hierarchical arrangement of data nodes arranged into one or more sub-trees. Each data node can be associated with a corresponding subset of the processed medical data from 402.

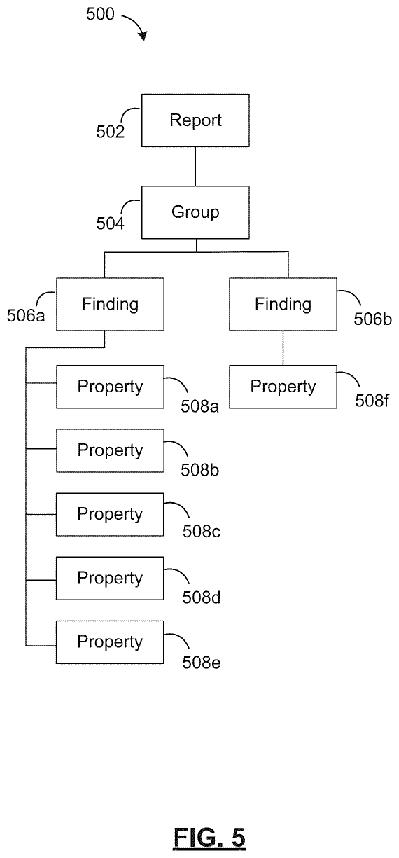

An example of a hierarchical tree structure 500 is shown in FIG. 5. As shown in FIG. 5, the hierarchical tree structure includes a top level parent or root node 502 for a given report. The highest-level node of the proposed report tree can be defined as the report node 502.

The report node 502 includes one or more child or branch nodes that define sub-trees dependent on the higher level report node 502. As shown in the example of FIG. 5, the report node 502 includes one or more group nodes 504 (also referred to as series nodes).

Optionally, the tree structure 500 may include one or more study nodes between the report node 502 and group nodes 504. Each study node corresponds to a particular image study for which processed medical data is contained within the report (and associated group nodes 504, finding nodes 506 and property nodes 508 nested therein). Each study nodes can be arranged between the report node 502 (i.e. nested within the report node 502) and one or more group nodes 504 nested within each study node.

The tree structure 500 may include a plurality of study nodes, e.g. where processed data corresponding to multiple studies is contained within a single report. Each study node can correspond to one of the studies associated with that report.

Alternatively, the study nodes may be omitted. This may be the case, for instance, where only a single image study is contained within a given report. Alternatively, the tree structure 500 may contain a study node even where the report corresponds to only a single image study. This may simplify processing by ensuring a consistent proposed report tree is displayed to a user.

Each group node 504 corresponds to a particular image series for which processed medical data is contained within the report (and associated finding nodes 506 and property nodes 508 nested therein). The tree structure 500 may include a plurality of group nodes 504, e.g. where processed data corresponding to multiple image series is contained within a single report. Each group node 504 can correspond to one of the series associated with that report.

Optionally, the group nodes 504 may be omitted. This may be the case, for instance, where only a single image series is contained within a given report.

Alternatively, the tree structure 500 may contain a group node 504 even where the report corresponds to only a single series. This may simplify processing by ensuring a consistent proposed report tree is displayed to a user.

Each group node 504 can contain one of more finding nodes 506a and 506b. Each finding node 506 contains the processed medical data associated with a particular clinical finding. Each clinical finding generally refers to the result or assessed outcome of a clinical investigation and can include data relating to observations, judgments or assessments of patients or patient-related data (including imaging data).

Each finding node can contain one or more property nodes 508a, 508b, 508c, 508d, 508e and 508f. Each property node 508 can include data relating to detailed information about the associated clinical finding (i.e. the clinical finding associated with the clinical finding node 506 within which that property node 508 is nested).

The processed medical data received at 402 can be mapped to the proposed report tree by mapping the processed medical data to different nodes of the tree structure 500.

The mapping of the processed medical data can be performed using an application-independent mapping. An application-independent mapping defines a set of rules for mapping processed medical data to the tree structure 500 regardless of the clinical application from which the processed medical data is received. That is, the same mapping rules or logic can be applied to processed medical data from different clinical applications. This may simplify mapping for clinical applications that output processed medical data with the same format and using the same output structure.

Alternatively, mapping of the processed medical data can be performed using an application-specific mapping. An application-specific mapping defines a set of rules for mapping processed medical data to the tree structure 500 that is specifically associated with the clinical application from which the processed medical data is received. Although an application-specific mapping is associated with a clinical application, it should be understood that the application-specific mapping for multiple clinical applications may ultimately be the same.

To map the processed medical data using an application specific mapping, the application specific mapping corresponding to the clinical software application (from which processed medical data is received at 402) is first determined. The application specific mapping can be determined by identifying the clinical software application that generated the set of processed medical data. The application specific mapping can then be selected from amongst a plurality of potential application specific mappings as the potential application specific mapping associated with the identified clinical software application

When mapping structured data (e.g. a structured report object such as object 600) to a hierarchical tree structure, the object nodes from the structured data can be identified. Each data node can then be associated with the proposed report tree concepts of report, group, finding and property (and optionally study). For example, the concepts associated with each data node can be identified from a defined report format, e.g. the format associated with the DICOM Structured Report object.

Alternatively or in addition, nodes can be identified and labelled based on the contents and properties of the labelled node (e.g. concept name, value type, etc.).

Alternatively or in addition, nodes can be identified and labelled based on their context within the processed medical data received at 402.

Each set of processed medical data can be mapped to a different corresponding proposed report tree. For example, each Structured Report object received at 402 can be mapped to a different corresponding proposed report tree such that n DICOM Structured Report objects are mapped to n proposed report trees. Similarly, each unstructured report object received at 402 can be mapped to a different corresponding proposed report tree.

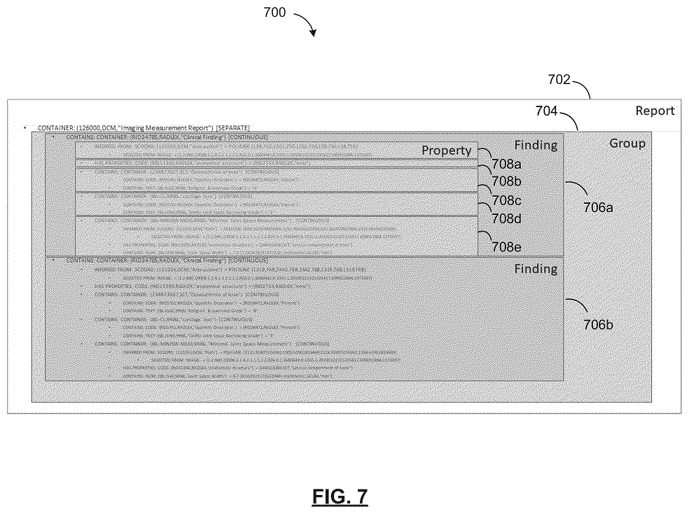

FIG. 7 illustrates an example mapping 700 of a Structured Report object into a proposed report tree. As shown in the example of FIG. 7, each portion of the processed medical data contained in Structured Report object 600 can be mapped to a corresponding node of a hierarchical tree structure.

The mapping 700 is determined based on the predefined format of the object 600. As shown in FIG. 7, the processed data contained in object 600 is mapped to a report node 702 that contains a single group node 704 corresponding to the image series associated with object 600. The group node 704 contains a pair of finding nodes 706a and 706b. Each finding node 706 includes a plurality of property nodes including property nodes 708a, 708b, 708c, 708d and 708e nested within finding node 706a.

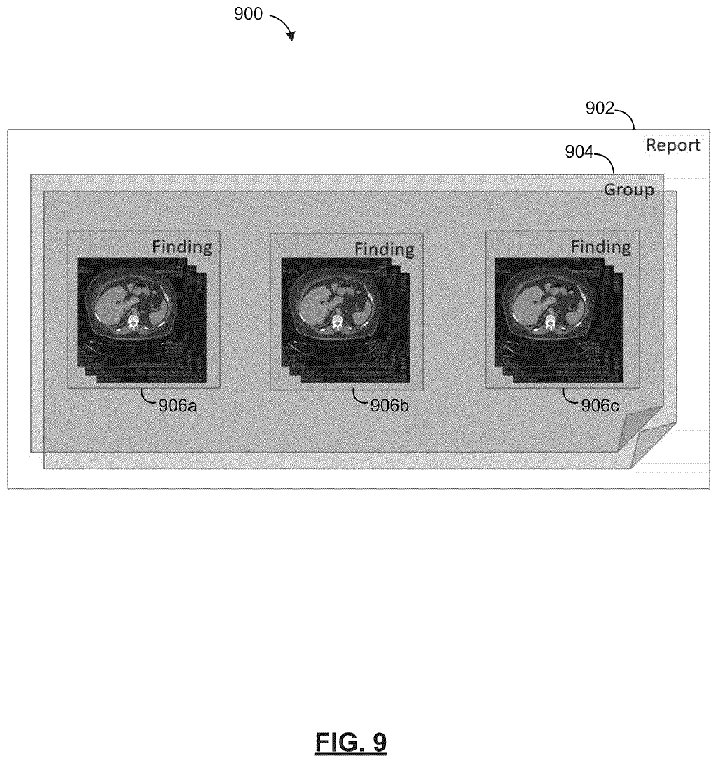

FIG. 9 illustrates an example mapping 900 of unstructured processed medical data into a proposed report tree. As shown in the example of FIG. 9, each portion of the processed medical data contained in unstructured data 800 can be mapped to a corresponding node of a hierarchical tree structure. This can allow unstructured objects to be organized into a defined report tree structure.

The mapping 900 can be determined based on the content of the processed medical data associated with a given unstructured DICOM object. The mapping 900 can define a top-level node shown as report node 902 that includes all of the processed data associated with the unstructured data 800. Each image series included in the unstructured data can be associated with a corresponding group node 904. Data associated with each image instance can be associated with a corresponding finding node 906a-906c. As shown in FIG. 9, the mapping 900 can omit property nodes e.g. where the properties associated with a given image instance are not identified in the unstructured data but are included directly in the image instance.

At 406, tagged nodes can generated for the proposed report tree. Each data node within the proposed report tree generated at 404 can be tagged with a corresponding semantic tag. Each semantic tag can specify a semantic meaning of a corresponding subset of the processed medical data associated with the corresponding tagged node. The semantic tags can be defined to provide a user an indication of the data contained or nested within each node. This may be particularly useful when presenting a visualization of a proposed report tree to a user through a viewer application. The semantic tags can provide a user with a clear understanding of the processed medical data contained within that node and facilitate review of a proposed report tree. The semantic tags can also indicate how the corresponding node should be displayed within the viewer application.

FIG. 10A illustrates an example graphical user interface of a proposed report tree that may be displayed to a user. As shown in FIG. 10A, a proposed report tree is displayed in the results area 1010 of the user interface 1000a. The proposed report tree includes a report node 1012, a study node 1018, and group nodes 1022a and 1022b.

As shown in FIG. 10A, the report node 1012 is tagged using a semantic tag (“riverrain-ClearReadCT”) that provides an indication of the data contained within that report. The study node 1018 also has a semantic tag applied (“Study Description—CHEST”) that provides an indication of the study data contained within that node. Each group node 1022 (or series node) also has a corresponding semantic tag that provides an indication of the data contained within that image series.

The semantic tags can be defined based on the node type (e.g. report node, study node, group node, finding node etc.), the content of the node (e.g. a CT scan, an MRI, a Chest study, a Head study, etc.), and/or a combination of the node type and the content (e.g. “Study Description—CHEST”).

Optionally, the semantic tags can be defined in different ways depending on whether the processed medical data is received as a structured object or an unstructured object. For example, semantic tags for all unstructured reports can be defined using a common tag definition algorithm while semantic tags for structured reports can be defined using application-specific tag definition algorithms. The application-specific tag definition algorithms can identify which portions of the data content can be used to define the semantic tags.

At 408, the proposed report tree can be displayed through a viewer application. The proposed report tree can be displayed through a viewer application on a display such as display 206 (see FIG. 2). Examples of graphical user interfaces that can be displayed through a viewer applications displaying proposed report trees are shown in FIGS. 10A-11 described in further detail herein below.

The proposed report tree can be displayed with at least some of the tagged nodes visible within the viewer application. The displayed nodes can be shown in the viewer application along with the semantic tags defined at 409. The viewer application can also visually indicate the relationship between the data nodes. For example, the viewer application can present labelled nodes with different visual appearances to indicate that a particular node (e.g. a finding node) is nested within a corresponding parent node (e.g. a group node 1022).

The viewer application can also present the proposed report tree with only a subset of the nodes displayed. For instance, where the proposed report tree includes at least one dependent node nested within a corresponding parent node within the hierarchical tree structure, the given proposed report tree can be displayed in the viewer application with the nested dependent node initially hidden. The graphical user interface can include a user-selectable input that can be used to render dependent nodes visible. This can provide a user with an easily navigable interface to review processed medical data in a structured and more easily digestible form.

The graphical user interfaces presented through the viewer application can include a variety of interactive elements to allow a user to easily navigate between and review different portions of the processed medical data. Examples of the interactive elements can include inputs usable to select and navigate between different proposed report trees, studies, and/or series; to expand and/or collapse dependent nodes within a given proposed report tree; to accept and/or reject data nodes within a given proposed report tree and so forth.

At 410, user input approving or rejecting at least a portion of the proposed report tree can be received. The user input can be received from the viewer application in response to the proposed report tree being displayed through the viewer application at 408. The user input can include an approval or rejection of at least a portion of the hierarchical tree structure.

The graphical user interfaces displayed at 408 can enable a user to accept or reject individual nodes or subtrees of the proposed report tree. The graphical user interface can include interactive elements enabling a user to accept or reject entire reports, studies, groups, findings and/or properties with a single action.

Referring to FIG. 10A, shown therein is a screenshot of an example graphical user interface 1000a that may be displayed through a viewer application. The example user interface 1000a shown in FIG. 10A shows an example of a proposed report tree generated from processed medical data received as a structured report. The graphical user interface 1000a provides an interactive interface to allow a user to review and accept/reject nodes of the proposed report tree.

The graphical user interface 1000a can include a plurality of panels or sections to facilitate navigating the processed medical data and thereby simplifying a user's review. In the example illustrated, the interface 1000a includes a series navigation panel 1002, a findings navigation panel 1006, a review input panel 1010, and a data viewer panel 1032 to allow a user to navigate and review a proposed report tree. The viewer application can also include report navigation elements to allow a user to navigate between different proposed report trees.

The series panel 1002 allows a user to navigate between different image series contained within a report. As shown, the series panel 1002 can include one or more series icons 1004a and 1004b corresponding to different image series contained in the processed medical data output by a clinical software application. In the example illustrated, a user can use the scroll bar on the right-hand side of the series panel 1002 to scroll through all the series generated by the clinical software application. The user can select a series icon 1004a or 1004b to view further information related to the series. Once selected, specific processed medical related to the selected series (i.e. contained within the corresponding series or group node) can be displayed through medical data viewer 1032. Each series icon 1004a and 1004b can be represented through a series title and a representative image. Series icons 1004a and 1004b can include further information about the series such as the number or images in the series.

The findings panel 1006 allows a user to navigate between different image series contained within a report. As shown, the findings panel 1006 can include one or more findings icons 1008a and 1008b. Each finding icon 1008a and 1008b shown corresponds to a lesion identified in a CT image.

In the example illustrated, a user can use the scroll bar on the right-hand side of the findings panel 1006 to scroll through all the findings contained in the processed medical data. The user can select a findings icon 1008a or 1008b to view further information related to the finding. Once selected, specific processed medical data related to the selected finding (i.e. contained within the corresponding finding node) can be displayed through medical data viewer 1032.

The medical data viewer 1032 can display the currently selected processed medical data. The currently selected processed medical data can include an image from the image study and other data related to the image. For example, the medical data viewer 1032 can display a CT image and a region of interest indicator 1042 that corresponds to a finding node. The region of interest indicator 1042 can include property data corresponding to the finding node. For example, the region of interest indicator 1042 can be a lesion indicator that displays the location, the length and the width of the lesion.

The review input panel 1010 can display information related to the proposed report tree. A user can provide input through the review input panel 1010 related to the report displayed in the graphical user interface 1000a. The review input panel 1010 can include interactive input elements corresponding to the nodes of the proposed report tree.

The review input panel 1010 can facilitate review of the proposed report tree by displaying the tagged nodes of the proposed report tree along with the associated semantic tags. Interactive input elements (also referred to as user input prompts) can be provided in association with each tagged node to enable a user to accept or reject the processed medical data contained within that node.

In the example illustrated, the review input panel 1010 includes a report node 1012, a report summary 1014, measurements 1016, a study node 1018 and associated study node input element 1020b, series nodes 1022a and 1022b and associated series node input elements 1026a and 1026b, comment field 1028 and report confirmation input element 1030.

The report node 1012 is a header that relates to all of the processed medical data in the report being reviewed in user interface 1000a. The report node 1012 is displayed along with the associated semantic tag defined for the report. As shown in user interface 1000a, navigation inputs allow a user to toggle between different reports by selecting the arrows at the right and left-hand sides of the report node 1012.

The report summary 1014 provides an overview of the processed medical data contained in the report. In the example illustrated, the report summary 1014 includes an overview of the total number of series in the report, the total number of findings in the report, the numbers of accepted and rejected series in the report and the numbers of accepted and rejected findings in the report.

Measurements 1016 can include processed medical data that is defined as geometric objects that are correlated to the data shown in viewer 1032. The measurements 1016 are examples of properties that have additional image-related semantic significance in that they can be displayed directly onto a corresponding image shown in the viewer 1032. That is, the measurements 1016 are contained within the properties node of a given report tree structure.

Study node 1018 includes all of the group nodes, finding nodes, and property nodes for a given study contained in the report. The study node 1018 can include an interactive element allowing a user to expand or collapse the processed medical data associated with the study node. This allows a user to select whether any dependent nodes nested within the study node 1018 are displayed in user interface 1000a. When the study node 1018 is toggled to a collapsed position, none of the sub-nodes (e.g. group nodes 1022) are shown in the user interface 1000a. When the study node 1018 is toggled to an expanded position, at least the next level of nodes (i.e. group nodes 1022a and 1022b) are shown in user interface 1000a as illustrated in FIG. 10A.

Group nodes (or series nodes) 1022a and 1022b can contain processed medical data for each of the series contained within the report. The group nodes 1022 can include an interactive element allowing a user to expand or collapse the processed medical data associated with the respective group node. This allows a user to select whether any dependent nodes nested within the group node 1022 are displayed in user interface 1000a. When a group node 1022 is toggled to a collapsed position (as shown in FIG. 10A), none of the sub-nodes (e.g. finding nodes 1036 shown in FIG. 10B) are shown in the user interface 1000a. When the group node 1022 is toggled to an expanded position, at least the next level of nodes (i.e. finding node 1036) are shown in user interface 1000a as illustrated in FIG. 10B.

As shown in FIG. 10B, finding nodes such as node 1036 can contain processed medical data for each finding contained within the report. The finding nodes 1036 can include an interactive element allowing a user to expand or collapse the processed medical data associated with the respective finding node. This allows a user to select whether any dependent nodes nested within the finding node 1022 (E.g. property nodes 1040a-1040c) are displayed in user interface 1000b. When a finding node 1036 is toggled to a collapsed position, none of the sub-nodes (e.g. property nodes 1040) are shown in the user interface 1000b. When the finding node 1036 is toggled to an expanded position, the next level of nodes (i.e. property nodes 1040) are shown in user interface 1000b as illustrated in FIG. 10B.

User interface 1000b includes an expanded finding node 1036 and property nodes 1040a, 1040b and 1040c. As shown, finding node 1036 is displayed with its associated semantic tag “Lesion 1 of 3”. Within the finding node 1036, multiple nested property nodes 1040a, 1040b and 1040c are shown. Property nodes 1040a, 1040b and 1040c can provide details about the corresponding parent finding node 1036. Property nodes 1040a, 1040b and 1040c can include information relating to the finding type, the location of the finding, finding size measurements, finding volume, density measurements, etc.

The user can toggle to different finding nodes (and associated properties) by selecting the arrows at the right and left-hand sides of the finding node 1036. Toggling to a different finding node can cause the medical data viewer 1032 and the findings panel 1006 to update to match the selection of the finding node 1036.

The user can also toggle the report node 1012 to view a different report, if available. Toggling to a different report node can cause the medical data viewer 1032, the series panel 1002 and the findings panel 1006 to update to match the selection of the report node 1012.

FIG. 10C shows another example user interface 1000c in which the selected series has been changed as compared to user interfaces 1000a and 1000b. Accordingly, the medical data viewer 1032 has been updated to display a different image series.

FIG. 10D shows another example user interface 1000d in which the selected series has been changed as compared to user interfaces 1000a, 1000b and 1000d. Accordingly, the medical data viewer 1032 has been updated to display a different image series.

The user interface 1000 displayed by the viewer application enables a user to easily accept or reject each data node within the hierarchical tree structure of a proposed report tree. As shown in FIGS. 10A-10D, each data note can have an associated node input element enabling a user to easily accept or reject the processed medical data contained within that node. The node input elements can also allow a user to accept or reject data from multiple nodes with a single input by allowing a user to accept or reject entire sub-trees or even the entire report.

The user can review a proposed report tree through the viewer application and interact with the input elements to approve or reject a report, study, group, finding and/or property nodes. As shown in FIGS. 10A-10D, the proposed report tree can be displayed within the viewer application user input prompts corresponding to different particular data nodes. Each user input prompt can enable a user to provide a corresponding input through the viewer application accepting or rejecting the definition of the corresponding data node.

A user input prompt can enable the user to accept or reject the definition of the corresponding particular data node and any portion of a sub-tree that is dependent on the corresponding particular data node. For example, a user can reject a finding and all property sub-nodes of that finding at the same time by rejecting only the parent finding. For instance, a user may accept or reject a study node 1018 (and all of the processed medical data contained within the sub-tree of nodes nested within the study node 1018) by selecting or deselecting the input element 1020b.

The user input prompts 1020a, 1020b, 1026a, 1026b, 1038 can be displayed as interactive checkboxes, as shown in the example of FIGS. 10A-10D. It should be understood that the user input prompts can include any user interface input control such as checkboxes, radio buttons, dropdown lists, list boxes, buttons, toggles and text fields for example.

The user prompt checkboxes can also provide a user with a visual indication of the current status of the data node and its related sub-nodes (e.g. accepted, rejected or partially accepted). An accepted user prompt (e.g. user prompt 1026b) can appear as a checkbox with a checkmark. A rejected user prompt (e.g. user prompt 1026a in FIG. 10A) can appear as a blank checkbox. A partially accepted user prompt (e.g. user prompts 1020a and 1020b) can appear as a partially filled checkbox indicating that only a portion of the sub-tree associated with that node has been accepted while another portion has been rejected. The user can click on or otherwise interreact with a checkbox to change the user input from accepted or rejected.

The user interface can also include a comment field 1028. The comment field 1028 can be a free-text input field that enables a user to insert comments in the proposed report tree. The comments can relate to any information the user wishes to store with the final report tree. For example, the comments can provide additional contextual information indicating why the report or portions of the report have been accepted/rejected. Examples of comments may include comments such as “Rejected lesion 2 because application measured incorrectly” or “Rejecting report because application incorrectly detected no lesions”. These contextual comments may assist subsequent reviewers and/or machine learning models in assessing the data that was accepted/rejected to refine data processing techniques and/or evaluate the correctness of the decision to accept or reject the data.

The user interface can also include a report tree finalization input element 1030 (“confirm choice” button). The report tree finalization input element 1030 allows a user to indicate that the proposed report tree has been reviewed and the user has completed selecting the nodes to be approved and/or rejected. The user can then select the report tree finalization input element 1030 to provide a report tree finalization input. The report tree finalization input can initiate a report finalization process to define the final report tree to be stored.

FIG. 10E illustrates an example user interface 1000e that may be presented to a user in response to the report tree finalization input. User interface 1000e illustrates an overview of the portions of the proposed report tree that are to be accepted and rejected for the final report tree. The user interface 1000e allows a user to review their inputs to ensure that they have reviewed all necessary portions of the proposed report tree and that the final report tree includes those portions the user has selected.

The example user interface 1000e include a final report overview 1050 that includes an accepted series overview 1052, accepted findings overview 1054, and rejected findings overview 1056, and a cancel input 1058 and confirm input 1060.

The final report overview 1050 displays an overview of the accepted and rejected nodes of the proposed report tree. The final report overview 1050 can display the accepted and rejected nodes in various ways, e.g. through list of the accepted reports, rejected reports, accepted groups (e.g. accepted series overview 1052), rejected groups, accepted findings (e.g. accepted findings overview 1054), rejected findings (e.g. rejected findings overview 1056), accepted properties and rejected properties.

The final report overview 1050 can also include additional information for the user relating to the report tree finalization process. For example, the final report overview 1050 1050 can specify the outcome of confirming review, where the final report tree will be stored and how the final report tree will be created.

The user can select cancel input 1058 to return to the viewer application displaying the proposed report tree and user inputs. Returning to the viewer application displaying the proposed report tree and user prompts will allow the user to continue editing the proposed report tree.

The user can select confirm input 1060 to confirm the accepted and rejected nodes. If the user selects the confirm button 1060, the accepted nodes of the proposed report tree node can be saved as the final report tree.

As noted above, a user may navigate to a different report node 1012 through the viewer application. FIG. 11 shows an example user interface 1100 that corresponds to the display of a different proposed report tree. In the example shown in FIG. 11, the proposed report tree is generated based on unstructured processed medical data. Similar to user interfaces 1000, the user interface 1100 includes a series panel 1102, a review input panel 1110 and a medical data viewer 1132.

The medical data viewer 1132 can display the currently selected processed medical data. As shown, the currently selected processed medical data can include a DICOM instance. For example, the medical data viewer 1132 can display a DICOM instance showing x-ray images with suspected fracture regions generated by a fracture detection clinical software application.

The review input panel 1110 can display information related to the proposed report tree. The results review panel 1110 can facilitate review of the proposed report tree by displaying the proposed report tree nodes and allowing the user to accept or reject the proposed report tree nodes. The results review panel 1110 can include a report node 1112, a report summary 1114, a study node 1118, a study node user input 1120, a series node 1122, a series node user input 1126, a comment field 1128 and a report tree finalization input element 1130 similar to those of user interface 1000.

At 412, a final report tree can be generated based on received user input. The final report tree can omit any portion of the hierarchical tree structure that was rejected by the user input. That is, the final report tree may only include those nodes that were accepted by the user through input to the viewer application.

The user input can include a report tree finalization user input as explained herein above. Each data node within the hierarchical tree structure can be accepted except for those data nodes that are explicitly rejected by the user. The rejected nodes that are removed from the final report tree can be deleted or stored in an archive. Optionally, accepted and rejected data can be tracked and archived to improve the report tree generation engine or the clinical software application.

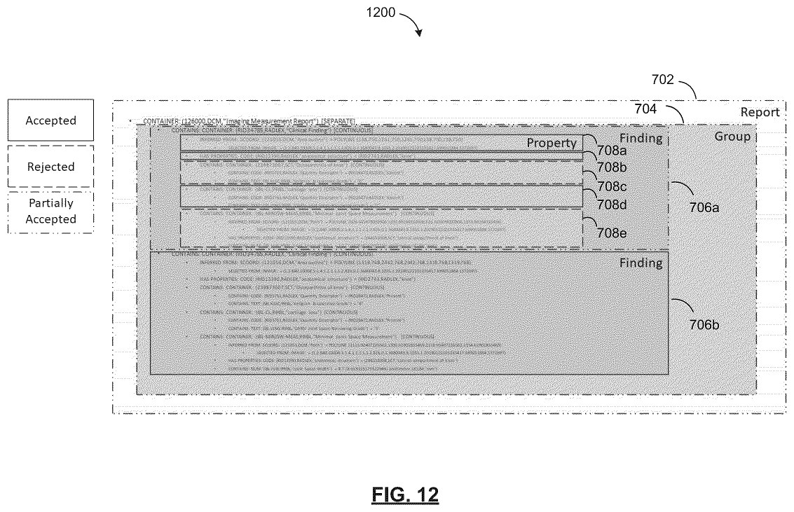

An example of a final report tree illustrating accepted and rejected data nodes is shown in FIG. 12. The final report tree illustrates a series of accepted and rejected nodes corresponding to the structured report object 700. As shown, the proposed tree corresponding to structured report object 700 includes a report node 702, a group node 704, finding nodes 706a and 706b and property nodes 708a, 708b, 708c, 708d and 708e.

As shown in FIG. 12, the report node 702, the group node 704 and the finding node 706a are partially accepted. The finding node 706b and the property nodes 708a, 708b and 708d are accepted. The property nodes 708c and 708e are rejected. The final report tree corresponding to report object 700 will thus include only the accepted and partially accepted data nodes.

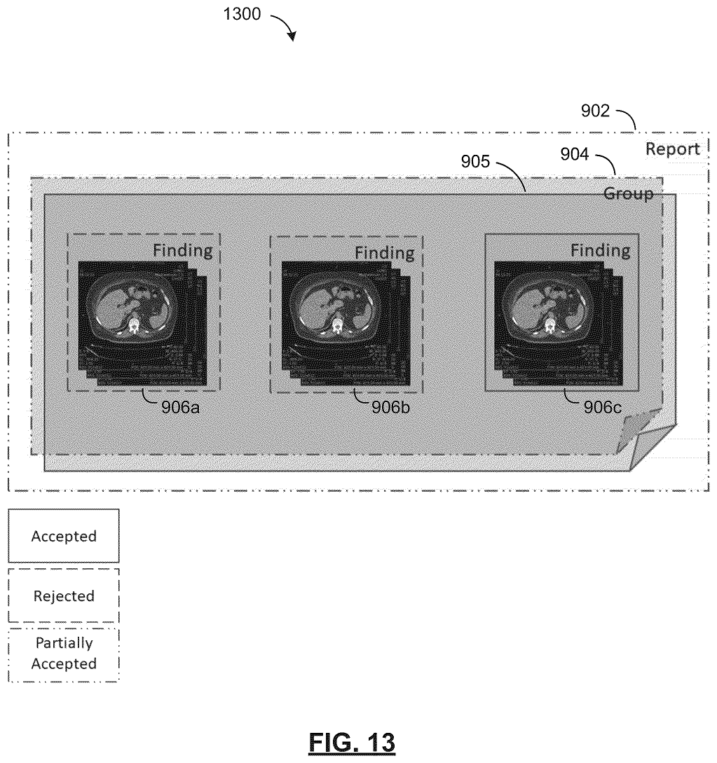

An example of a final report tree illustrating accepted and rejected data nodes is shown in FIG. 13. The final report tree illustrates a series of accepted and rejected nodes corresponding to the unstructured report object 900. As shown, the proposed tree corresponding to unstructured report object 900 includes a report node 902, group nodes 904 and 905 and finding nodes 906a, 906b and 906c.

As shown in FIG. 13, report node 902 and the group node 904 are partially accepted. The group node 905 and the finding node 906c are accepted. The finding nodes 906a and 906b are rejected. The final report tree corresponding to unstructured report object 900 will thus include only the accepted and partially accepted data nodes.

At 414, the final report tree containing the processed medical data can be stored in an electronic data management system. The processed medical data stored in the electronic data management system can omit the processed medical data that was rejected by the user input. For structured final report trees, a final DICOM Structured Report object can be generated using the final report tree. The final DICOM Structured Report object can be stored in the electronic data management system. Storing an unstructured final report tree can involve storing the unstructured DICOM objects corresponding to the final report tree in the electronic data management system. The unstructured objects referred to in both an unstructured final report tree and a structured final report tree can be stored in the electronic data management system.

The final report tree can be transmitted and/or stored using various data formats. In some cases, the final report tree can be stored directly in the data management system in the original report format. For example, the final report tree can be stored in a PACS in a DICOM format.

Alternatively or in addition, the final report tree can be converted into an alternative format prior to being transmitted to, or stored by, an electronic data management system. For example, the final report tree can be converted into one or more report messages defined in a format compatible with the electronic data management system. For example, the report message(s) can be formatted according to the HL7 v2.x standard or converted into FHIR objects. Alternatively or in addition, the report message(s) can be defined to call proprietary APIs exposed by the electronic data management system (e.g. a reporting system or RIS).

Optionally, unstructured objects may be embedded within the corresponding final report tree. This may allow the unstructured data to be transmitted to the electronic data management system as an image embedded within a report. For example, some electronic data management systems that use DICOM formats (e.g. PACS) and/or other data formats (e.g. reporting systems and RIS) may support the embedding of image data within reports. Accordingly, the report message(s) corresponding to the final report tree can be defined to incorporate the unstructured data (e.g. as embedded image data).

Machine learning techniques may be applied to improve the mapping between Structured and unstructured objects and the proposed report trees. For example, annotated report trees can be stored and analyzed to identify any patterns or trends relating to the data mapping. The training algorithm may incorporate data reflecting accepted and rejected data nodes to determine patterns within the annotated data labelling. This training process may generate a model that can automatically determine the relevant parts of the annotated reports and generate a new annotated report only containing the relevant data, eliminating the need for the user/technician to manually accept and/or reject segments of the annotated reports. Various different learning techniques may be applied, such as a supervised learning system wherein a user/technician alters the data.

While the above description provides examples of one or more processes or systems, or computer program products, it will be appreciated that other processes or systems, or computer program products may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for processing medical data received from a clinical software application, the method comprising:
receiving, at a processor, a set of processed medical data generated by the clinical software application, wherein the set of processed medical data is generated by the clinical software application based on at least one medical image generated by an enterprise imaging device and associated image data corresponding to the at least one medical image, wherein the at least one medical image is associated with a medical image study;
mapping, by the processor, the set of processed medical data to at least one proposed report tree, wherein each proposed report tree is defined with a hierarchical tree structure that includes a plurality of data nodes arranged into one or more sub-trees, wherein each data node is associated with a corresponding subset of the processed medical data;
for each proposed report tree, generating a plurality of tagged nodes by tagging, by the processor, at least some of the data nodes within that proposed report tree with a corresponding semantic tag, wherein each semantic tag specifies a semantic meaning of a corresponding subset of the processed medical data associated with the corresponding tagged node;
displaying, by the processor, a given proposed report tree from the at least one proposed report tree through a viewer application, wherein the given proposed report tree is displayed with at least some of the tagged nodes visible within the viewer application;
receiving, by the processor, user input through the viewer application in response to the proposed report tree being displayed through the viewer application, wherein the user input includes an approval or rejection of at least a portion of the hierarchical tree structure;
generating, by the processor, a final report tree based on the received user input; and
storing, by the processor, the processed medical data contained within the final report tree in an electronic data management system.

2. The method of claim 1, wherein the final report tree omits any portion of the hierarchical tree structure that was rejected by the user input whereby the processed medical data stored in the electronic data management system omits the processed medical data that was rejected by the user input.

3. The method of claim 1, wherein the user input defines the approval or rejection of each data node within the hierarchical tree structure.

4. The method of claim 3, wherein the user input includes a report tree finalization input, and each data node within the hierarchical tree structure is accepted except for those data nodes that are explicitly rejected by a user.

5. The method of claim 1, wherein:
the given proposed report tree comprises at least one dependent node nested within a corresponding parent node within the hierarchical tree structure; and
when the given proposed report tree is displayed in the viewer application the nested dependent node is initially hidden.

6. The method of claim 5, wherein the given proposed report tree is displayed within the viewer application with a user-selectable input usable to render the nested dependent node visible.

7. The method of claim 1, wherein the given proposed report tree is displayed within the viewer application with at least one user input prompt, wherein each user input prompt corresponds to a particular data node, and each user input prompt enables a user to provide a corresponding input through the viewer application accepting or rejecting the definition of the corresponding particular data node.

8. The method of claim 7, wherein a given user input prompt enables the user to accept or reject the definition of the corresponding particular data node and any portion of a sub-tree that is dependent on that corresponding particular data node.

9. The method of claim 1, wherein the set of processed medical data generated by the clinical software application comprises at least one DICOM Structured Report object, and each DICOM Structured Report object is mapped to a different corresponding proposed report tree.

10. The method of claim 1, wherein the set of processed medical data generated by the clinical software application comprises a DICOM Structured Report object, and the plurality of data nodes are identified from a defined report format associated with the DICOM Structured Report object.

11. The method of claim 1, wherein storing the processed medical data contained within the final report tree in the electronic data management system comprises:
generating a final DICOM Structured Report object using the final report tree; and
storing the final DICOM Structured Report object in the electronic data management system.

12. The method of claim 1, wherein:
the set of processed medical data corresponding to the given proposed report tree comprises one or more unstructured DICOM objects; and
storing the processed medical data contained within the final report tree in the electronic data management system comprises storing the unstructured DICOM objects corresponding to the final report tree in the electronic data management system.

13. The method of claim 1, wherein:
the set of processed medical data comprises one or more unstructured DICOM objects;
the one or more unstructured DICOM objects are mapped to data nodes within the proposed report tree using a hierarchical structure that includes a plurality of node levels, the plurality of node levels including a study node level, a series node level, and an instance node level; and
each data node is tagged based on the node level that data node is mapped to.

14. The method of claim 1, wherein
the set of processed medical data comprises one or more unstructured DICOM objects; and
each unstructured DICOM object is mapped to a corresponding data node within the proposed report tree based on the content of the processed medical data associated with that unstructured DICOM object.

15. The method of claim 14, wherein mapping the set of processed medical data to the at least one proposed report tree comprises:
mapping the set of processed medical data to the hierarchical tree structure using an application independent mapping.

16. The method of claim 1, wherein mapping the set of processed medical data to the at least one proposed report tree comprises:
determining an application specific mapping corresponding to the clinical software application; and
mapping the set of processed medical data to the hierarchical tree structure according to the application specific mapping.

17. The method of claim 16, wherein the application specific mapping is determined by:
identifying the clinical software application that generated the set of processed medical data; and
selecting the application specific mapping from amongst a plurality of potential application specific mappings, wherein the application specific mapping is selected as the potential application specific mapping associated with the identified clinical software application.

18. The method of claim 17, further comprising modifying the application specific mapping based on the received user input.

19. A system for processing medical data received from a clinical software application, the system comprising:
a network device coupled to an electronic data management system; and
one or more processors in communication with the network device, the one or more processors configured to:
receive, using the network device, a set of processed medical data generated by the clinical software application, wherein the set of processed medical data is generated by the clinical software application based on at least one medical image generated by an enterprise imaging device and associated image data corresponding to the at least one medical image, wherein the at least one medical image is associated with a medical image study;
map the set of processed medical data to at least one proposed report tree, wherein each proposed report tree is defined with a hierarchical tree structure that includes a plurality of data nodes arranged into one or more sub-trees, wherein each data node is associated with a corresponding subset of the processed medical data;
for each proposed report tree, generate a plurality of tagged nodes by tagging at least some of the data nodes within that proposed report tree with a corresponding semantic tag, wherein each semantic tag specifies a semantic meaning of a corresponding subset of the processed medical data associated with the corresponding tagged node;
display a given proposed report tree from the at least one proposed report tree through a viewer application, wherein the given proposed report tree is displayed with at least a given proposed report tree from the at least one proposed report tree through the viewer application, wherein the given proposed report tree is displayed with at least some of the tagged nodes visible within the viewer application;
receive user input through the viewer application in response to the proposed report tree being displayed through the viewer application, wherein the user input includes an approval or rejection of at least a portion of the hierarchical tree structure;
generate a final report tree based on the received user input; and
store, using the network device, the processed medical data contained within the final report tree in the electronic data management system.

20. The system of claim 19, wherein the one or more processors are configured to generate the final report tree to omit any portion of the hierarchical tree structure that was rejected by the user input whereby the processed medical data stored in the electronic data management system omits the processed medical data that was rejected by the user input.

21. The system of claim 19, wherein the user input defines the approval or rejection of each data node within the hierarchical tree structure.

22. The system of claim 21, wherein the user input includes a report tree finalization input, and each data node within the hierarchical tree structure is accepted except for those data nodes that are explicitly rejected by a user.

23. The system of claim 19, wherein:
the given proposed report tree comprises at least one dependent node nested within a corresponding parent node within the hierarchical tree structure; and
the one or more processors is configured to display the given proposed report tree in the viewer application with the nested dependent node initially hidden.

24. The system of claim 23, wherein the one or more processors is configured to display the given proposed report tree through the viewer application with a user-selectable input usable to render the nested dependent node visible.

25. The system of claim 19, wherein the one or more processors is configured to display the given proposed report tree through the viewer application with at least one user input prompt, wherein each user input prompt corresponds to a particular data node, and each user input prompt enables a user to provide a corresponding input through the viewer application accepting or rejecting the definition of the corresponding particular data node.

26. The system of claim 25, wherein a given user input prompt enables the user to accept or reject the definition of the corresponding particular data node and any portion of a sub-tree that is dependent on that corresponding particular data node.

27. The system of claim 19, wherein the set of processed medical data generated by the clinical software application comprises at least one DICOM Structured Report object, and the one or more processors is configured to map each DICOM Structured Report object to a different corresponding proposed report tree.

28. The system of claim 19, wherein the set of processed medical data generated by the clinical software application comprises a DICOM Structured Report object, and the one or more processors is configured to identifying the plurality of data nodes from a defined report format associated with the DICOM Structured Report object.

29. The system of claim 19, wherein the one or more processors is further configured to store the processed medical data contained within the final report tree in the electronic data management system by:
generating a final DICOM Structured Report object using the final report tree; and
storing the final DICOM Structured Report object in the electronic data management system.

30. The system of claim 19, wherein:
the set of processed medical data corresponding to the given proposed report tree comprises one or more unstructured DICOM objects; and
the one or more processors is further configured to store the processed medical data contained within the final report tree in the electronic data management system by storing the unstructured DICOM objects corresponding to the final report tree in the electronic data management system.

31. The system of claim 19, wherein:
the set of processed medical data comprises one or more unstructured DICOM objects; and
the one or more processors is further configured to:
map the one or more unstructured DICOM objects to data nodes within the proposed report tree using a hierarchical structure that includes a plurality of node levels, the plurality of node levels including a study node level, a series node level, and an instance node level; and
tag each data node based on the node level that data node is mapped to.

32. The system of claim 19, wherein:
the set of processed medical data comprises one or more unstructured DICOM objects; and
the one or more processors is further configured to map each unstructured DICOM object to a corresponding data node within the proposed report tree based on the content of the processed medical data associated with that unstructured DICOM object.

33. The system of claim 32, wherein the one or more processors is further configured to map the set of processed medical data to the at least one proposed report tree by:
mapping the set of processed medical data to the hierarchical tree structure using an application independent mapping.

34. The system of claim 19, wherein the one or more processors is further configured map the set of processed medical data to the at least on proposed report tree by:
determining an application specific mapping corresponding to the clinical software application; and
mapping the set of processed medical data to the hierarchical tree structure according to the application specific mapping.

35. The system of claim 34, wherein the one or more processors is further configured to determine the application specific mapping by:
identifying the clinical software application that generated the set of processed medical data; and
selecting the application specific mapping from amongst a plurality of potential application specific mappings, wherein the application specific mapping is selected as the potential application specific mapping associated with the identified clinical software application.

36. The system of claim 35 wherein the one or more processors is further configured to modify the application specific mapping based on the received user input.

37. A non-transitory computer-readable medium with instructions stored thereon for processing medical data received from a clinical software application, that when executed by a processor, performs the method of claim 1.
